(19) United States
(12) Patent Application Publication
(10) Pub. No.: US 2023/0167443 A1
(43) Pub. Date: Jun. 1, 2023

(54) Optimised Methods for Cleavage of Target Sequences

(71) Applicant: Cancer Research Technology Limited, London (GB)

(72) Inventor: Iain Alasdair RUSSELL, Cambridge (GB)

(21) Appl. No.: 17/911,184

(22) PCT Filed: Mar. 16, 2021
(86) PCT No.: PCT/GB2021/050650

§ 371 Date: Sep. 13, 2022

(30) Foreign Application Priority Data
Mar. 16, 2020 (GB) 2003814.7

(51) Int. Cl. C12N 15/113; C12N 15/10; C12N 9/22

(52) U.S. Cl. CPC C12N 15/113; C12N 15/102; C12N 9/22; C12N 2310/20; C12N 2320/11

(57) ABSTRACT

The invention provides methods of selecting guide RNA sequences, and the use of such sequences in CRISPR-Cas gene editing of a target sequence. In particular, the invention relates to a method of selecting guide RNA sequences, based on the determined frequencies of editing outcomes, which in one case result in low mosaicism and in another case result in large deletions or knockouts.

The present invention relates to a method of selecting a site in a target nucleic acid sequence for cleavage, for example by an endonuclease. The invention provides methods of selecting guide RNA sequences, and the use of such sequences in CRISPR-Cas gene editing of a target sequence. In particular, the invention relates to a method of selecting sites for cleavage, for example by selecting guide RNA sequences, based on the determined frequencies of editing outcomes.

Gene editing may be carried out using nucleases to introduce breaks in the nucleic acid sequence of interest; during the repair of those breaks, the natural repair processes may introduce errors in the sequence and thereby edit it. For example, one such nuclease system, CRISPR-Cas9 gene editing, has revolutionised genetically modified animal production worldwide. However, cell populations and genetically modified animals produced using nucleases, such as CRISPR methodologies, are mosaic, containing different genetic edits at the intended target site throughout the cell population or tissues of the animal. Mosaicism stems from semi-random repair that occurs after the nuclease, such as Cas9, identifies and cleaves its intended target DNA. In addition, genome editing that occurs in a multi-cell population, such as after the one-cell stage embryo, further contributes to mosaicism, as the newly formed edits will not be homogenously distributed throughout the cell population, e.g. animal. In short, mosaicism occurs because the repair of an individual double-stranded DNA break (DSB) is an independent process with a probabilistic outcome. Thus, the chance of multiple DSBs being repaired in a similar manner, either between different alleles in a single cell, or in difference cells, is on average very low. Mosaic cell populations or animals cannot be used in experiments as the genetic impurity will lead to confounded data. Mosaicism in animals is removed through multiple rounds of breeding and back-crossing to generate mice with pure edits throughout the animal. This is a costly and time-consuming process, as for every new mouse model produced, on average 170 mice are wasted. In other species, such as non-human primates and livestock, the process of breeding out mosaicism will take years. In the context of cell populations, mosaicism is removed through the process of single cell cloning and expansion of individual clones.

Known strategies for reducing mosaicism revolve around limiting the total number of independent edits that occur. In the context of an embryo, this means editing at the one-cell stage where the number of total edits is limited, usually one edit per allele in a normal cell (i.e. two edits in a diploid organism). To achieve this in a cellular population, either singly isolated cells can be edited, or single-cell derived cloning is required from the edited population. In both contexts, editing needs to be constrained to the one cell stage. Approaches attempted thus far focus on containing or constricting mosaicism. For example, by speeding up the process of editing (e.g. using in vitro transcribed gRNAs and recombinant Cas9 protein rather than having the editing components encoded in messenger (m)RNA or DNA plasmids which require the cellular processes of transcription and translation before editing can occur); by editing earlier in the one-cell phase (e.g. by introducing CRISPR-Cas9 components into very early stage zygotes, particularly those produced by in vitro fertilization (IVF) to allow editing to ‘complete’ earlier, before the two-cell stage, or by using alternative microinjection protocols (Lamas-Torazo et al., Nature Scientific Reports, Volume 9, Article number: 14900 (2019)); by actively cutting short the editing window (e.g. by accelerating the degradation of the Cas9 endonuclease to avoid editing in the two-cell stage); or by editing before the one-cell stage (e.g. germline modification by editing spermatogonial stem cells, oocytes or haploid ESCs); or finally, by enhancing the efficiency of precise genome editing (e.g. by using long ssDNA as repair donors over conventional, less efficient means) (Mehravar et al., Dev Biol. 2019 Jan. 15; 445(2): 156-162).

Generally, these strategies focus on limiting the ‘activity window’ of the nuclease so that all editing occurs at the one-cell stage or within a single cell. While this means that the total number of independent editing events are reduced, the issue of how a given DSB is repaired, which is fundamental to mosaicism, is not addressed. As such, none of these existing approaches will ever broadly eliminate mosaicism, other than by accidental chance.

The problem of mosaicism is not restricted to the creation of transgenic animals, but is also an issue in therapeutic contexts where differing mutations in a population or pool of cells may have different phenotypic consequences (e.g. in-frame deletions or unintended gain-of-function mutations). Consequently, to ensure homogeneity of the edited cell pool (i.e. the same editing outcome in every cell), the pools must be single cell cloned and then individual clones expanded. This is an extremely resource-intensive process, and furthermore is not compatible with many primary cell types, for example T cells. This presents a significant hurdle in the production of certain therapies, such as CAR-T cell production, and may be integral to the safety profiles of such medicines.

The present invention has been devised with these issues in mind.

According to a first aspect, the invention provides methods of identifying sites, or target sequences, for cleavage in a nucleic acid sequence. The sites may be considered optimised cleavage sites, for example for better controlling uniformity of edited sequences and/or for reducing mosaicism of a population of edited cells. The nucleic acid sequence may comprise, for example, a gene sequence. The cleavage may be by a nuclease that may cause a double-stranded break, for example a blunt-ended double-stranded break, in the nucleic acid sequence.

The method comprises:
identifying a plurality of target sequences in a nucleic acid sequence, wherein the target sequences may be targeted for cleavage, for example by a nuclease;
determining the frequency of editing outcomes for each of the plurality of target sequences; and
selecting one or more target sequences which are predicted to result in a major editing outcome following cleavage.

The methods may be of particular use in optimising CRISPR-Cas systems of gene editing. In these instances, the target sequences may be understood to be defined by the guide RNA sequences used in the CRISPR-Cas systems, due to the guide RNA sequences binding the target sequences and thereby targeting them for cleavage by the Cas endonuclease. Thus, there is provided a method of selecting one or more guide RNA sequences for use in CRISPR-Cas editing of a nucleic acid sequence, the method comprising:
identifying a plurality of guide RNA sequences which target the nucleic acid sequence;
determining the frequency of editing outcomes for each of the plurality of guide RNA sequences; and
selecting one or more guide RNA sequences which are predicted to result in a major editing outcome.

In some embodiments of methods of the invention, the step of selecting one or more target (e.g. guide RNA) sequences which are predicted to result in a major editing outcome comprises selecting one or more target (e.g. guide RNA) sequences for which the frequency of the most abundant (i.e. major) editing outcome is determined to be at least 2-fold greater than the frequency of the second most abundant editing outcome.

In some embodiments, the methods of the invention may comprise the step of selecting more than one guide RNA sequence for use in CRISPR-Cas9 editing of more than one nucleic acid sequence. In such an embodiment, suitably more than one nucleic acid sequence may be targeted and edited. Suitably more than one nucleic acid sequence may be edited in the same method, suitably simultaneously. In such embodiments, the method may comprise a step of identifying a plurality of guide RNA sequences which target a plurality of nucleic acid sequences. Suitably such an embodiment may be referred to as stacking of guide RNA sequences.

As used herein, the term “editing outcome” refers to the genotype (i.e. the DNA sequence) resulting from the editing process, e.g. CRISPR-Cas9 editing process.

It will be appreciated that, in the following, where a “guide RNA sequence” or similar is referred to it may equally apply to a target sequence similarly and correspondingly identified as a preferred site for targeted nucleic acid cleavage. Thus, where a “guide RNA sequence” is referred to in combination with the CRISPR-Cas enzyme or system for which it is designed, this may equally be considered to refer to a corresponding “target sequence” and associated nuclease that will cleave it.

In some embodiments, the method comprises selecting one or more target or guide RNA sequences for which the frequency of the most abundant (i.e. major) editing outcome is determined to be at least 2-fold, for example at least 3-fold, at least 4-fold, at least 5-fold, at least 6-fold, at least 8-fold, at least 10-fold, at least 12-fold, at least 15-fold or at least 20-fold, greater than the frequency of the second most abundant editing outcome.

The methods of the invention are thus based on a selection process which maximises the difference between the major (most abundant) genotype frequency and the second most abundant genotype frequency. This can be calculated using the following equation: (Frequency of the most abundant (major) editing outcome)/(Frequency of the second most abundant editing outcome).

For example, the major (most abundant) editing outcome which results from CRISPR-Cas editing of a target sequence using a given guide RNA sequence may be determined (e.g. predicted) to be a 7-base-pair deletion, having a frequency of 54.4%. The second most abundant editing outcome may be determined (e.g. predicted) to be a 1-base pair insertion of a cytosine nucleotide, having a frequency of 4.3%. In this case, the frequency of the most abundant editing outcome is 12.7 fold (54.4/4.3) greater that the frequency of the second most abundant editing outcome.

In preferred methods the most abundant editing outcome (genotype frequency) will be a desired outcome, e.g. a particular frameshift mutation as explained further below. Thus the methods, by determining selections based on major editing outcomes, may maximise the fold change difference between a desired outcome and the second most likely outcome of the edit.

The present inventors have been the first to utilise the fold change metric when selecting target sequences for cleavage, for example when designing guide RNA sequences for CRISPR-Cas gene editing and, in particular, the first to apply this metric in the selection of guide RNA sequences for reducing or eliminating mosaicism in a cell population, such as a multi-cell organism. The present inventors have appreciated that reducing or eliminating mosaicism requires not only the same editing outcome to occur on each allele in a single cell, but also on each allele across multiple cells. The use of fold change allows the reliable generation of a homogenously edited population of cells, which in turn enables mosaicism to be reduced or eliminated.

The frequency of editing outcomes for each of the plurality of guide RNA sequences may be determined using a computer model (e.g. a machine learning algorithm). The computer model may be configured to predict the editing outcomes, and the relative frequency of each outcome, for a given guide RNA sequence. Suitable computer models include FOREcasT (Allen, Nature Biotechnology, Volume 37, Pages 64-72, 2019), inDelphi (Shen et al., Nature volume 563, page 646, 2018) and Lindel (Nucleic Acids Research, Volume 47, Pages 7989-8003, 2019). The FOREcasT model is available as a webtool (https://www.forecast.app) or can be run locally (e.g. using R programming language). The inDelphi model is also available via a webtool (available at https://indelphi.giffordlab.mit.edu/) or it can be run locally (e.g. in Python programming language). The Lindel model is also available as a webtool (https://lindel.gs.washington.edu/Lindel/docs/) or can be run locally (e.g. using Python programming language). Additionally, the Lindel model has been adapted into the CRISPOR guide design tool (available at http://www.crispor.org).

Without wishing to be bound by any theory, the inventors have identified that the extent of microhomology around a cleavage site plays an important role in determining how cleavage at that site will be repaired. Significant information is now available, in the form of computer models such as those identified above, regarding how CRISPR-Cas9 cleavage of alternative target sites will be repaired, and that information can be used to predict and be selective about desired editing outcomes, according to the methods of the invention. However, since the nature of the cleavage sites are important for determining how cleavage at those sites will be repaired, the methods of the invention may also relate to cleavage carried out by alternative methods, for example using alternative nucleases including TALENs or ZFNs. In such methods using alternative nucleases, computer models based on CRISPR-Cas9 cleavage, such as are described above, may, for example, be used to identify target sequences having major editing outcomes and then, instead of cleaving those sequences using a CRISPR-Cas9:guide RNA system, the sequences may be cleaved using alternative nucleases, for example TALENs or ZFNs designed to target those sequences. It will be appreciated that determining how a given target sequence will be repaired can also be empirically determined through direct experimentation in cells by targeting these sequences for cleavage, and sequencing the editing outcomes.

Thus, in some embodiments, the method comprises predicting, using a computer model, the editing outcomes of each of the plurality of target sequences or guide RNA sequences. The method and associated calculations using a computer model may be carried out on one or more computers in a single location, for example on a desktop computer or server in a single location, or, alternatively, the method and associated calculations using a computer model may be carried out across different locations, for example using the internet or carrying out calculations on servers based in the cloud. The benefit of computer models, such as machine learning tools, is that they speed up the selection of target sequences or guide RNAs that will yield a desirable pattern of repair outcomes. However, it will be appreciated that the same outcome could be achieved, for example, empirically by editing cells at multiple target sequences or using a plurality of guide RNA sequences which target the nucleic acid sequence of interest, sequencing the resulting DNA to determine the spectrum of editing outcomes, and choosing target sequences or guides based on the sequencing output. It is appreciated that the spectrum of editing outcomes will generally be highly similar between cells of different origins. In cell types where differences in DNA repair pathway fidelity exist, for example where key DNA repair genes are mutated or upon perturbation by chemical means, it will be appreciated that DNA repair outcomes may need to be determined in the particular cell type under investigation.

Thus, in some embodiments, the step of determining the frequency of editing outcomes for each of a plurality of target sequences comprises: carrying out editing at each of the plurality of the target sequences using a nuclease of interest; and sequencing the DNA resulting from each editing process. Similarly, the step of determining the frequency of editing outcomes for each of a plurality of guide RNA sequences may comprise: carrying out CRISPR-Cas editing of the nucleic acid sequence using each of the plurality of guide RNA sequences; and sequencing the DNA resulting from each editing process.

As is known in the art, mosaicism stems from the action of cellular mechanisms which operate to repair the double-strand break (DSB) following cleavage of DNA by an endonuclease such as a Cas endonuclease. In the absence of a donor template, these repair mechanisms (which include non-homologous end joining (NHEJ) and microhomology-mediated end joining (MMEJ)) are imperfect and often lead to a deletion or insertion of one or more nucleotides (referred to collectively as ‘indels’), resulting in mutations. A cut site is not always repaired in the same way, such that cleavage of a given site can give rise to different genotypes which appear with different relative frequencies. Guide RNAs are known to produce characteristic patterns of editing outcomes following Cas cleavage of their target site. These patterns are non-random, and the same distribution of edits will normally arise from a given guide sequence, no matter what cells the guide is used in. For example, a given guide RNA sequence may be found to result in a 7-base pair deletion in 40% of editing outcomes, a 1-base pair deletion in 20% of editing outcomes, a 1-base pair deletion in 20% of editing outcomes, a 2-base pair insertion in 10% of editing outcomes and either alternative or no editing outcomes in the remaining 10%. Accordingly, when the CRISPR-Cas editing is applied to a population of cells, the repair process may result in different mutations in different cells, producing a mosaic of editing outcomes across the population. It has recently been found that the errors in DSB repair are influenced by the microhomology of the DNA sequence around the cut site. DNA sequences with low homology are generally repaired to give a wide spectrum of editing outcomes, while areas of high homology will generally repair to a narrower spectrum of editing outcomes. The present inventors have surprisingly found that this finding can be harnessed to reduce or eliminate mosaicism.

Thus, by maximising the ratio between the frequency of the most abundant (major) genotype (i.e. editing outcome) and the frequency of the second most abundant genotype (i.e. editing outcome), a single major editing outcome may be achieved.

The existing approaches to reducing mosaicism fail to take into account the local DNA architecture and features in the DNA that dictate how a given DSB will be repaired. These approaches focus on the timing of nuclease action and the generation of the DSBs, rather than on the resolution of DSBs, which is informed by local DNA architecture and features. In contrast, the invention focuses on how the DSBs are resolved, based on the understanding that the resolution of DBSs is influenced by local DNA features. The invention enables more independent DSBs to be formed and repaired in the same manner (either within the same cell or in separate cells). In practical terms, having control over how DSBs are repaired means that editing is no longer restricted to single cell populations, such as the one-cell stage of embryo development or a singly isolated cell.

The plurality of guide RNA sequences which target the nucleic acid sequence may be identified by any suitable technique known to those skilled in the art. Potential CRISPR-Cas target regions (and thus corresponding guide sequences) may be identified by proximity to a protospacer adjacent motif (PAM). For example, all possible guide RNA sequences which target a given gene may be identified using publicly available software, such as UCSC Genome Browser, Deskgen, CRISPOR or Lindel. Similarly, possible target sequences may be identified according to the characteristics of the cleavage mechanism, e.g. the nuclease used for cleavage.

In some embodiments, the method comprises identifying a plurality of target sequences or guide RNA sequences which target the coding sequence of a gene.

As an alternative to eliminating mosaicism, the finding that local homology affects the editing outcome can also be harnessed to implement large deletions or ‘knock-outs’. Instead of choosing a gRNA which target areas of high microhomology to ensure a narrow spectrum of editing and reduce mosaicism, the inventors have found that choosing pairs of gRNAs which target regions of low microhomology can be used to implement deletions. Such deletions can be used to excise parts of gene sequences and produce knock-out models which are equally as useful as the models with reduced mosaicism. Such a method is described in the second aspect of the invention herein.

In some embodiments therefore, the method comprises identifying a plurality of target sequences or guide RNA sequences which target the non-coding sequence of a gene. For example, in some cases it may be desirable to target the introns either side of an exon, so as to excise the entire exon to cause a knockout. The methods may comprise targeting intergenic regions or other non-coding ‘genes’, for example miRNAs or other non coding RNA classes (lncRNA, snoRNA, piRNAs). The methods may comprise targeting key regulatory elements, for example enhancer regions.

Prior to identifying the target sequences or guide RNA sequences, the method may further comprise identifying the primary transcript(s) of a gene to be targeted. The primary transcript(s) of a given gene may be determined using publicly available genomics tools, such as Ensembl.

In some embodiments, the method further comprises selecting the target sequences or guide RNA sequences which target (i.e. are complementary to) a region located in the first 40%-70% or the first 50-60% of a gene (or the coding sequence thereof). Target sequences or guide RNA sequences which target the remaining portion of the gene may be excluded.

In some embodiments, the step of selecting target sequences or guide RNA sequences which target the first 40%-70% (e.g. the first about 50%) of the gene (or coding sequence thereof) may conveniently be carried out prior to the step of determining the editing outcomes of the target sequences or guide RNA sequences. Targeting the upstream portion (e.g. the first half) of the gene increases the likelihood of eliminating the key functional domains of the protein encoded by the gene.

In some embodiments the method further comprises selecting the target sequences or guide RNA sequences which are determined or predicted to result in a frameshifting mutation. Because proteins are encoded from triplets of RNA/DNA, there is a one in three chance that an edit will be a multiple of three, in which case the frame of the gene will not be changed. This potentially results in the expression of a functional protein. It may therefore be advantageous to select target sequences or guide RNAs which cause frameshifting, such that the DNA downstream of the cut site is out of frame with the original sequence.

Frameshifting can be selected for by selecting sequences for which the most abundant (i.e. major) editing outcome is not determined or predicted to be an insertion or a deletion of a number of nucleotides which is a multiple of three.

It will be appreciated that it may be desirable to avoid frameshifting. For example, it may be desired to delete several amino acids from a protein in order to destroy its function. Thus, in some embodiments the method comprises selecting target sequences or guide RNA sequences which are determined or predicted to avoid a frameshifting mutation. A non-frameshifting mutation can be selected for by selecting target sequences or guide RNA sequences for which the most abundant editing outcome is determined or predicted to be an insertion or a deletion of a number of nucleotides which is a multiple of three.

In some embodiments, the method may comprise assigning each of the target sequences or guide RNA sequences a frameshifting score, using a computer model. The sequences with the most desirable frameshifting scores may then be selected.

For example, the computer model Lindel can be used to determine the “frameshift %” score for a given guide RNA sequence. The frameshift % score indicates the probability that the edit will result in either a non-frameshifting mutation, a frameshift of 1 nucleotide, or a frameshift of 2 nucleotides. For example, a ratio of +0=33.3%; +1=33.3%; +2=33.3% would mean there is an equal chance of the edit moving the sequence either in-frame, 1 base pair out of frame, or 2 base pairs out of frame respectively. In some embodiments, the method comprises selecting the guide RNAs for which the non-frameshift % score is less than about 33% (e.g. less than 33.3%), such that the selected guide RNAs are predicted to bias the outcome towards a frameshifting edit. In some other embodiments, e.g. if an in-frame deletion is desired, the method may comprise selecting the guide RNAs for which the non-frameshift % score is more than about 33% (e.g. more than 33.3%).

In some embodiments, the method further comprises excluding any target sequences or guide RNA sequences which target orphan exons that are not present in all major transcripts. In eukaryotic cells, some genes have multiple transcripts that do share all of the exons. Therefore, in embodiments in which it is desired to create a knock-out of a gene of interest in a eukaryotic cell, to ensure that expression of the gene is completely eliminated it may be advantageous to target an area of the gene that is common to all transcripts. An exception to this is where it is desired to knock out a specific isoform that is characterised by the presence of an orphan exon, thus some embodiments may comprise selecting target sequences or guide RNA sequences that promote the exclusion of an orphan exon, or part thereof, from the transcript of a gene.

In some embodiments the method further comprises assigning each target sequence or guide RNA sequence an off-target score, and excluding any sequences with a score below a predetermined threshold. This helps to avoid undesired editing of the genome at sites other than the target sequence.

The target sequences and guide RNA sequences may be assigned an off-target score using a computer model or algorithm. Suitable models will be known to those skilled in the art, and include UCSC Genome Browser, CRISPOR, and Deskgen. These models are based on the algorithm described by Hsu et al., Nature Biotechnology volume 31, pages 827-832 (2013).

In some embodiments, each guide RNA sequence is assigned an off-target score from 1 to 100, wherein a score of 1 represents many hundreds or thousands of off-targets and a score of 100 represents no off-targets. The method may comprise excluding guide RNA sequences having a score of less than 80, less than 70, less than 60, less than 50 or less than 40. The off-target score may be calculated using a computer model or algorithm as described herein.

In some embodiments the method further comprises assigning each target sequence or guide RNA sequence an on-target activity score, and excluding any sequences with a score below a predetermined threshold. On-target activity scores are used to predict how well a guide sequence is likely to cut at a given site.

The guide RNA sequences may be assigned an on-target activity score using a computer model or algorithm, for example on a web platform. Suitable web platforms will be known to those skilled in the art, and include UCSC Genome Browser, CRISPOR, Deskgen. Suitable models may be based on the metric described by Doench et al., Nature Biotechnology volume 34, pages 184-191(2016) or by Moreno-Mateos et al, Nature Methods volume 12, pages 982-988 (2015).

In some embodiments, each guide RNA sequence is assigned an on-target activity score of from 1 to 100, wherein a score of 100 represents the highest predicted activity based on nucleotide sequence and a score of 1 represents the lowest predicted activity. The method may comprise excluding any guide RNA sequences with a score of less than 50, less than 40, less than 30, or less than 20. The on-target score may be calculated using a computer model or algorithm as described herein.

A method using guide RNA sequences in accordance with the first aspect of the invention may comprise all of the following steps, or any combination thereof: selecting the guide RNA sequences which target a region located in the first 40-70% (e.g. 50%) of a gene of interest (or, optionally, the coding sequence thereof); selecting the guide RNA sequences which are determined or predicted to result in or avoid a frameshifting mutation; excluding any guide RNA sequences which target orphan exons that are not present in all major transcripts; assigning each guide RNA sequence an off-target score, and excluding any guide RNA sequences with a score below a predetermined threshold; and assigning each guide RNA sequence an on-target activity score, and excluding any guide RNA sequences with a score below a predetermined threshold. It will be appreciated that these steps may be carried out in any order. Some or all of these steps may be carried out before or after the step of determining the frequency of editing outcomes for each of the plurality of guide RNA sequences. It will further be appreciated that each step carried out may result in the exclusion of some guide RNA sequences from analysis in subsequent steps. Accordingly, not all of the guide RNA sequences identified as targeting the gene or coding sequence thereof will necessarily be analysed in each step of the method. The number of potential guide RNA sequences analysed may decrease with each additional step carried out.

In some embodiments, the method of selecting one or more guide RNA sequences for use in CRISPR-Cas editing of a gene comprises:
identifying a plurality of guide RNA sequences which target the gene (or the coding sequence thereof);
optionally, selecting the guide RNA sequences which target a region located in the first ˜50% of the gene (or coding sequence thereof)—in these embodiments, guide RNA sequences which target the second ˜50% of the gene are excluded from subsequent analysis;
optionally, excluding any guide RNA sequences which target orphan exons that are not present in all major transcripts;
determining the frequency of editing outcomes for each of the plurality of guide RNA sequences; and
selecting one or more guide RNA sequences for which the frequency of the most abundant (i.e. major) editing outcome is determined to be at least 2-fold greater than the frequency of the second most abundant editing outcome;
optionally, selecting the guide RNA sequences which are determined or predicted to result in a frameshifting mutation (guide RNA sequences which are determined or predicted to result in an in-frame mutation being excluded from subsequent analysis);
optionally, assigning each guide RNA sequence an off-target score, and excluding any guide RNA sequences with a score below a predetermined threshold; and
optionally, assigning each guide RNA sequence an on-target activity score, and excluding any guide RNA sequences with a score below a predetermined threshold.

In a second aspect, methods of the invention may be used to design improved systems for generating deletions of stretches of DNA between two target sites. Such methods may comprise choosing guide RNAs for CRISPR-Cas systems as above that target the 5′ and 3′ flanks of a DNA sequence intended for deletion, but identifying guides with a large number of editing outcomes (such that the sequences targeted for cleavage are generally characterised by low microhomology), such that the cleavage will be preferentially repaired by deletion of the intervening DNA sequence between the two cleavage sites. The target sequences flanking the DNA to be deleted may be separated by a distance of either greater than 20 bp, 200 bp, 2000 bp, or greater than 2 Mb.

In such improved methods for deleting large stretches of DNA sequences, the most abundant outcome from cleavage of a target sequence may be less than 4 fold, less than 3 fold, less than 2 fold, less than 1.5 fold greater than that of the second most abundant outcome, and the frequency of the most abundant outcome may be less than 2 fold greater than the third, fourth or fifth most abundant outcome, for example less than 2.5 fold greater, less than 3 fold greater, or less than 4 fold greater. In one embodiment, the frequency of the most abundant outcome may be less than 2 fold greater than the second most abundant outcome. In one embodiment, the frequency of the most abundant outcome may be less than 2 fold greater than the third most abundant outcome.

Those methods may further comprise assigning each guide RNA sequence an off-target score, and excluding any guide RNA sequences with a score below a predetermined threshold, for example excluding any guide RNA sequences with an off-target score of less than 50, less than 40, less than 30, or less than 20. Furthermore, those methods may comprise assigning each guide RNA sequence an on-target score, and excluding any guide RNA sequences with a score below a predetermined threshold, for example excluding any guide RNA sequences with an on-target score of less than 80, less than 70, less than 60, less than 50, less than 40 or less than 30. Of course, the frameshifting score and position of the cleavage site within the first half of a gene may be less important in such methods for deleting large stretches of DNA sequences.

Therefore, in one embodiment, the invention may comprise a method of selecting a pair of guide RNA sequences for use in CRISPR-Cas editing of a nucleic acid sequence, the method comprising:
identifying a plurality of guide RNA sequences which target the 5′ and 3′ flanks surrounding the nucleic acid sequence;
determining the frequency of editing outcomes for each of the plurality of guide RNA sequences; and
selecting a pair of guide RNA sequences comprising a first guide RNA which targets the 5′ flank and a second guide RNA which targets the 3′ flank, wherein for each guide RNA the frequency of the most abundant editing outcome is determined to be less than 4 fold greater than the frequency of the second most abundant editing outcome.

In one embodiment, the method is a method of selecting a pair of guide RNA sequences for use in CRISPR-Cas deletion of a nucleic acid sequence. Suitably, the nucleic acid sequence is intended to be deleted.

In one embodiment, the method comprises identifying a plurality of guide RNA sequences which target the 5′ flank of the nucleic acid sequence and identifying a plurality of guide RNA sequences which target the 3′ flank of the nucleic acid sequence.

In a further embodiment, the invention comprises a method for editing a nucleic acid sequence in an organism, a cell or a population of cells, or in a cell-free expression system, the method comprising exposing double-stranded (dsDNA) comprising the nucleic acid sequence to a Cas endonuclease and a pair of guide RNA molecules which are capable of directing the Cas endonuclease to target the 5′ and 3′ flanks surrounding the nucleic acid sequence, wherein the pair of guide RNA molecules comprises a first guide RNA and a second guide RNA which, when used in CRISPR-Cas editing, result in (or are predicted to result in, e.g. by a computer model), a major editing outcome having a frequency which is less than 4 fold greater than the frequency of the second most abundant editing outcome.

In one embodiment, both guide RNA molecules result in (or are predicted to result in, e.g. by a computer model), a major editing outcome having a frequency which is less than 4 fold greater than the frequency of the second most abundant editing outcome.

In one embodiment, the nucleic acid sequence is exposed to more than one Cas endonuclease, suitably at least two Cas endonucleases. In some embodiments, the nucleic acid sequence may be exposed to a plurality of Cas endonucleases, for example within a cell.

Suitably, the pair of guide RNA molecules are capable of directing the or each Cas endonuclease to target and cleave the 5′ and 3′ flanks surrounding the nucleic acid sequence.

Suitably the pair of guide RNA molecules are capable of directing the or each Cas endonuclease to target and cleave the 5′ and 3′ flanks surrounding the nucleic acid sequence so as to produce two double strand breaks. Suitably the double strand breaks are produced in the 5′ and 3′ flanks surrounding the nucleic acid sequence. Suitably the double strand breaks are produced on either side of the nucleic acid sequence.

Suitably the nucleic acid sequence is removed. Suitably the nucleic acid sequence is removed after the or each Cas endonuclease targets and cleaves the 5′ and 3′ flanks surrounding the nucleic acid sequence.

In one embodiment, the method is a method for deleting a nucleic acid sequence in an organism, a cell, or a population of cells, or in a cell-free expression system.

Suitably the nucleic acid sequence in such embodiments is a sequence which it is desirable to delete. Suitably the sequence which it is desirable to delete can be any sequence. Suitably the sequence may be in a coding region or a non-coding region. Suitably the sequence may comprise the whole or a part of a gene sequence, or a regulatory element. Suitable regulatory elements include cis or trans regulatory elements. Suitable cis regulatory elements that may be deleted include nucleic acid sequences encoding: enhancers, silencers, promoters, insulators. Suitable trans regulatory elements that may be deleted include nucleic acid sequences encoding: transcription factors, siRNA, lncRNA, miRNA. RNP, SR proteins, DNA editing proteins.

In one embodiment, the nucleic acid sequence which it is desirable to delete comprises an exon. Suitably the exon may be a coding exon. Suitably, the exon may be a ‘critical exon’.in which the removal of said exon will cause a frameshift in the coding sequence of that gene. Suitably in such embodiments, the pair of gRNAs direct the or each Cas endonuclease to target the 5′ and 3′ flanks surrounding the critical exon. Suitably, a critical exon refers to one or more exons, which when removed disrupt the codon phasing in the rest of the nucleic acid sequence, causing a frameshift mutation to occur. Suitably the resulting frameshift mutation results in disruption of the coding sequence of the rest of the nucleic acid.

In one embodiment, such sequences may include deleterious or pathological nucleic acid sequences. Suitably such sequences may include nucleic acid sequences which encode a molecule which causes or is involved in a disease. For example, the nucleic acid sequence may encode a mutant form of a protein which causes a genetic disorder, or the nucleic acid sequence may encode an enhancer element which acts to increase expression of a protein resulting in a genetic disorder. Alternatively, the sequence may include a nucleic acid sequence where its deletion is of interest for research. Suitably, deletion of the nucleic acid sequence causes a disease. Suitably by deleting such a sequence, a disease model is created.

In one embodiment, such sequences may be endogenous or exogenous to the cell or organism to be modified. Suitably the nucleic acid sequence which it is desirable to delete may be exogenous to the cell or organism to be modified. Suitably the exogenous nucleic acid sequence may be a transgenic or heterologous nucleic acid sequence. Suitably in such embodiments, a heterologous or transgenic nucleic acid sequence may have been integrated into the DNA by a previous process, and it is desirable for it to be removed at a later stage.

Suitably the first guide RNA which targets the 5′ flank targets a sequence within the 5′ flank, and suitably the second guide RNA which targets the 3′ flank targets a sequence within the 3′ flank. By ‘5′ flank’ it is meant the nucleotide sequence before the nucleic acid sequence to be deleted. By ‘3′ flank’ it is meant the nucleotide sequence after the nucleic acid sequence to be deleted. Suitably in order from 5′ to 3′. Suitably immediately before or immediately after. Suitably, the 5′ flank and the 3′ flank may be considered to comprise up to 1 kb, up to 500 bp, up to 400 bp, up to 300 bp, up to 200 bp, up to 100 bp, up to 50 bp, up to 40 bp, up to 30 bp, up to 20 bp, up to 10 bp from the 5′ and the 3′ end of the nucleic acid sequence respectively. Suitably, the sequence targeted by the first guide RNA is within a 5′ flank comprising up to 1 kb, up to 500 bp, up to 400 bp, up to 300 bp, up to 200 bp, up to 100 bp, up to 50 bp, up to 40 bp, up to 30 bp, up to 20 bp, up to 10 bp from the 5′ end of the nucleic acid sequence. Suitably, the sequence targeted by the second guide RNA is within a 3′ flank comprising up to 1 kb, up to 500 bp, up to 400 bp, up to 300 bp, up to 200 bp, up to 100 bp, up to 50 bp, up to 40 bp, up to 30 bp, up to 20 bp, up to 10 bp from the 3′ end of the nucleic acid sequence. Suitably, the 5′ flank and the 3′ flank may be adjacent to either end of the nucleic acid sequence, suitably adjacent to the 5′ and the 3′ end of the nucleic acid sequence respectively.

The nucleic acid sequence to be deleted may be greater than 20 bp, 200 bp, 2000 bp, or greater than 2 Mb in length.

In one embodiment, the 5′ flank and the 3′ flank comprise sequences with low microhomology. In one embodiment, the first guide RNA targets a sequence of low microhomology in the 5′ flank. In one embodiment, the second guide RNA targets a sequence of low microhomology in the 3′ flank.

In one embodiment, for each guide RNA the frequency of the most abundant editing outcome is determined to be less than 4 fold greater, less than 3 fold greater, less than 2.5 fold greater, less than 2 fold greater, less than 1.5 fold greater than the frequency of the second most abundant editing outcome. In one embodiment, for each guide RNA the frequency of the most abundant editing outcome is determined to be about equal to the frequency of the second most abundant editing outcome.

In one embodiment, for each guide RNA the frequency of the most abundant editing outcome is determined to be less than 4 fold greater, less than 3 fold greater, less than 2.5 fold greater, less than 2 fold greater, less than 1.5 fold greater than the frequency of any other editing outcome. In one embodiment, for each guide RNA the frequency of the most abundant editing outcome is determined to be about equal to the frequency of any other editing outcome.

The invention also provides systems designed according to this second aspect.

In a third aspect, methods of the invention may be used to design improved systems for incorporating heterologous sequences into a stretch of target DNA, i.e. they may be used for “knock in” experiments. Such methods may comprise: (i) choosing guide RNAs for CRISPR-Cas systems as above that target a DNA sequence, but identifying guides with a large number of editing outcomes (such that the sequences targeted for cleavage are generally characterised by low microhomology), and (ii) engineering microhomology into each end of the donor sequence that is to be introduced into the target region, to create artificial regions of high microhomology between the cut site and the knock-in template, such that the cleavage will be preferentially repaired by incorporation of the donor sequence.

In such improved methods for incorporating heterologous sequences into a stretch of target DNA the frequency of the most abundant outcome may be less than 1.5 fold greater than that of the second most abundant outcome, and the frequency of the most abundant outcome may be less than 2 fold greater than the fifth most abundant outcome, for example less than 2.5 fold greater, less than 3 fold greater, or less than 4 fold greater. Those methods may further comprise assigning each guide RNA sequence an off-target score, and excluding any guide RNA sequences with a score below a predetermined threshold, for example excluding any guide RNA sequences with an off-target score of less than 50, less than 40, less than 30, or less than 20. Furthermore, those methods may comprise assigning each guide RNA sequence an on-target score, and excluding any guide RNA sequences with a score below a predetermined threshold, for example excluding any guide RNA sequences with an on-target score of less than 80, less than 70, less than 60, less than 50, less than 40 or less than 30. Of course, the frameshifting score and position of the cleavage site within the first half of a gene may be less important in such methods for incorporating heterologous sequences into a stretch of target DNA.

To bias the integration of the donor nucleic acid molecule at the DSB over indel formation, artificial microhomology may be engineered into the donor molecule. The sequence of the donor molecule may be altered to include di-nucleotide, tri-nucleotide, or longer stretches of microhomology, that are within 30 bp upstream (5′) or downstream (3′) of the DSB. Microhomology stretches may be incorporated in any position within the donor molecule. These methods may further comprise the inclusion of microhomology regions that preserve the coding sequence of a gene in which they are incorporated. In these instances, there will be no unintentional disruption of the protein sequence, other than intentional changes purposefully introduced by the donor sequence (for example, disease causing mutations, activating mutations or inactivating mutations).

The newly formed sequence at the cut site, comprised of a flank of native DNA and a flank of DNA with artificially engineered microhomology will, if cleaved, be predicted to generate a major editing outcome that is 2-fold or greater than the second most predicted editing outcome in the same manner as stated above. To determine the predicted editing outcome of an engineered microhomology stretch, a computer model, such as Lindel, can be used. It will be appreciated that this can also be empirically determined through direct experimentation in cells by introducing the engineered microhomology into cells, targeting this for cleavage, and sequencing the editing outcomes.

The invention also provides systems designed according to this third aspect.

In some embodiments the methods of the invention further comprise generating a guide RNA molecule comprising a guide RNA sequence selected using the methods described herein. The method of selecting guide RNA sequences described herein may result in a number of guide RNA sequences which meet the criteria applied in the selection process and which could potentially be used in CRISPR-Cas gene editing. Therefore, in some embodiments, the method may comprise generating multiple (e.g. 2, 3, 4, 5, 8, 10 or more) guide RNA molecules. The guide RNA molecules may then be tested.

In some embodiments, the method further comprises testing one or more guide RNA molecules comprising the selected guide RNA sequence(s) to determine the editing outcome(s) i.e. the genotypes which result from CRISPR-Cas editing of the target sequences. A guide RNA molecule may be tested by carrying out CRISPR-Cas editing of the target sequence using the guide RNA molecule (for example in a suitable cell line, e.g. mouse ES cells), and then sequencing the edited sequence.

The CRISPR-Cas gene editing and/or subsequent sequencing may be carried out using the protocols described herein. Following the editing process, genomic DNA may be extracted from the cells using standard techniques. The region surrounding the target locus may be amplified prior to sequencing, for example using PCR. Sequencing may be carried out using any suitable technique, such as Sanger sequencing. Sequencing data may be analysed using software, for example the Sanger sequencing trace deconvolution webtool (ICE, available from Synthego), in order to determine the editing outcomes of each guide RNA molecule tested. The frequency of each genotype generated for each guide RNA molecule may thus be determined. The editing efficiency obtained with each guide RNA molecule may also be assessed, i.e. the percentage of the total number of DNA molecules that are edited at the predicted cleavage site. Preferably, the editing efficiency of guide RNA molecules selected for further use will be at least 10%, at least 20%, at least 30%, at least 40%, at least 50%, at least 60%, at least 70%, at least 80%, at least 90% or at least 100%. A cut off of 30% editing efficiency means 30% of all available target sites are edited in the target DNA, for example in the embryo or the cell pool examined.

Preferred methods of the invention comprise assessing and selecting target sequences or guide RNAs according to the number and frequency of genotypes that are produced using them, as detailed above, followed by selection of those target sequences or guide RNAs having a 25% editing efficiency. The selection of guide RNAs having at least 25% editing efficiency is particularly preferred in methods of generating non-human animal models, for example mouse models.

Following testing, the selected guide RNA molecule may be used to edit a cell (e.g. a zygote) or a population of cells. Following testing, the selected target sequence may be targeted by a nuclease to edit a cell (e.g. a zygote) or a population of cells.

It will be understood that the term “guide RNA molecule” refers to a nucleic acid molecule that is capable of forming a complex with a CRISPR-Cas endonuclease and direct sequence-specific binding of the complex to a target nucleic acid sequence. The guide RNA molecule comprises the guide RNA sequence (which may also be referred to as the “targeting sequence”) selected using the methods described herein. In some embodiments the guide RNA molecule may be chemically modified or nucleic acid analogues. The guide RNA may comprise RNA and/or DNA sequences.

Guide RNA molecules can be generated using techniques commonly known to those skilled in the art. For example, guide RNA molecules can be generated using chemical synthesis. Another method is to use in vitro transcription in which the guide RNA molecule is transcribed using a DNA template. Alternatively, the guide RNA molecule may be expressed by a vector, such as a plasmid or viral vector, which has been transfected into a host cell.

In some embodiments, the guide RNA molecule is a single guide RNA (sgRNA). The term “single guide RNA” refers to a single RNA molecule for use in a CRISPR-Cas9 system which comprises the crRNA sequence (which comprises the targeting sequence) fused to the scaffold tracrRNA sequence. However, it will be appreciated that the invention may also be implemented using a dual molecule crRNA:tracrRNA system, or a system that uses a non-traditional tracrRNA sequence.

It will be appreciated by those skilled in the art that a “target” (also referred to in the art as a “target locus”) of a guide RNA sequence is a region of a nucleic acid sequence to which a molecule comprising the guide RNA sequence is capable of binding (“hybridizing”) e.g. through Watson-Crick base-pairing. The ability of a guide RNA sequence to bind to its target may be described with reference to the level of complementarity between the guide RNA sequence and the target sequence. The level of complementarity can be expressed as a percentage identity between the guide RNA sequence and its target sequence, the percentage identity being the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% identity).

A guide RNA sequence must have sufficient complementarity to its target nucleic acid sequence to hybridize with the target nucleic acid sequence. Thus, in some embodiments, the degree of complementarity between a guide RNA sequence and its corresponding target sequence may be at least about 50%, 60%, 75%, 80%, 85%, 90%, 95%, 97%, 98%, 99%, 99.5% or 100%. A greater degree of complementarity may be preferred in order to reduce the off-target score of the RNA sequence, and a greater degree of complementarity may be required in particular regions of the RNA sequence, for example in the region proximal to the PAM sequence. Alignment between a guide RNA sequence and its target sequence may be determined using, for example, any of the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

According to a fourth aspect of the present invention there is provided a method for editing a nucleic acid sequence in an organism, a cell or a population of cells, or in a cell-free expression system. The method may comprise exposing double-stranded (ds)DNA comprising the nucleic acid sequence to a nuclease that is targeted to a target sequence within the nucleic acid sequence that is predicted to result in a major editing outcome following cleavage. The target sequence may be, for example, within a target gene or a non-coding region, as explained above. The target sequence may be selected using the methods described above, and thus the methods of this fourth aspect may further comprise steps or methods of the first aspect.

Preferably the nuclease is a Cas endonuclease, such as a Cas9 endonuclease, and the Cas endonuclease is targeted by a guide RNA molecule which is capable of directing the Cas endonuclease to the target sequence, for example of a target gene. The guide RNA may be selected using the methods described above, thus the methods of the fourth aspect that involve the use of guide RNA sequences may further comprise steps or methods of the first aspect that concern guide RNA sequences.

A method according to the fourth aspect may comprise using a system designed according to the second or third aspect, and thus may comprise steps or methods of the second or third aspect.

A fourth aspect of the invention provides a method for editing a nucleic acid sequence in an organism, a cell or a population of cells, or in a cell-free expression system, the method comprising exposing double-stranded (dsDNA) comprising the nucleic acid sequence to a Cas endonuclease and a guide RNA molecule which is capable of directing the Cas endonuclease to the target sequence within the nucleic acid sequence.

In some embodiments, the guide RNA molecule comprises a guide RNA sequence which, when used in CRISPR-Cas editing, results in (or is predicted to result in, e.g. by a computer model), a major editing outcome having a frequency which is at least 2-fold, at least 3-fold, at least 4-fold, at least 5-fold, at least 6-fold, at least 8-fold, at least 10-fold, at least 12-fold, at least 15-fold or at least 20-fold greater than the second most abundant editing outcome. Alternatively, in some embodiments, the guide RNA molecule when used in CRISPR-Cas editing, may result in a major editing outcome with an abundance of less than 4 fold greater than the other editing outcomes as described in the second aspect.

The guide RNA molecule may have been generated according to the methods described herein. The guide RNA molecule may comprise a guide RNA sequence selected according to the methods described herein.

In one embodiment, as mentioned herein above, more than one guide RNA may be used in such a method of the fourth aspect. Suitably therefore, the method of the fourth aspect may comprise a method for editing more than one nucleic acid sequence in an organism, a cell or a population of cells, or in a cell-free expression system.

In one embodiment, therefore, there is provided a method for editing more than one nucleic acid sequence in an organism, a cell or a population of cells, or in a cell-free expression system, the method comprising exposing double-stranded (dsDNA) comprising each nucleic acid sequence to a Cas endonuclease and more than one guide RNA molecule, wherein each guide RNA molecule is capable of directing the Cas endonuclease to a target sequence within one of the nucleic acid sequences.

For example, there is provided a method for editing two nucleic acid sequences in an organism, a cell or a population of cells, or in a cell-free expression system, the method comprising exposing double-stranded (dsDNA) comprising the first and second nucleic acid sequences to a Cas endonuclease and two guide RNA molecules, wherein the first guide RNA molecule is capable of directing the Cas endonuclease to a target sequence within the first nucleic acid sequence, and the second guide RNA molecule is capable of directing the Cas endonuclease to a target sequence within the second nucleic acid sequence.

The method may be for repressing the expression of a target gene, for example by creating a knock-out mutation, e.g. a frameshift mutation. Suitably this may be achieved by deletion of a critical exon.

In embodiments in which the method is for editing the target sequence in a cell or population of cells, the method may comprise introducing the guide RNA molecule and the DNA endonuclease into the cell or cells. In embodiments in which the method is for editing more than one target sequence in a cell or population of cells, the method may comprise introducing more than one guide RNA molecule and optionally more than one DNA endonuclease into the cell or cells.

The guide RNA molecule and the Cas endonuclease may be introduced into a cell, or into each cell within a population, separately (either sequentially or simultaneously) or in combination. For example, the guide RNA molecule and the Cas endonuclease may be provided in a single composition for administration to the cell(s). Introduction of the guide RNA molecule and Cas endonuclease into a cell may be performed via viral vectors known to the skilled person e.g., lentiviral vector, adenoviral vector, AAV vector.

The guide RNA molecule and the Cas endonuclease may be introduced into the cell(s) by any suitable technique. Such techniques will be known to those skilled in the art, and include lipofection, viral vectors (such as Lentiviral or Adeno-associated Virus vectors), virus-like particles, nanoparticles, electroporation, nucleofection, microinjection and other means of transfection or transduction.

In some embodiments, the guide RNA molecule and the Cas endonuclease are introduced into the cell(s) by electroporation. The guide RNA molecule and Cas endonuclease may be complexed prior to electroporation. Suitable electroporation methods are known in the art and may be further described herein.

Suitable nucleases for use in methods of the invention include Class II CRISPR-Cas systems. Preferably, the methods described herein may utilise or be configured for nucleic acid sequence editing using a CRISPR-Cas system, for example a CRISPR-Cas system belonging to Class II, in particular Class II B (e.g. Cas9), or Class V-A (e.g. Cas12a).

In some embodiments, the Cas endonuclease cleaves the target sequence so as to produce a blunt-ended double strand break. In other embodiments the Cas endonuclease will cleave the target sequence so as to produce a staggered double strand break, with an overhang at the break site of less than 8 nucleotides, for example less than 6 or less than 4 or less than 2 nucleotides.

In preferred embodiments the Cas endonuclease is a Cas9 endonuclease. For example, the Cas9 may be a naturally occurring Cas9 isolated from Streptococcus pyogenes (SpCas9). In some embodiments, the Cas endonuclease is a variant or homologue of a naturally occurring Cas9, having at least 70%, at least 80%, at least 85%, at least 90%, at least 95%, at least 97%, at least 98% or at least 99% identity to a naturally occurring Cas9, such as SpCas9.

Other Cas9 endonucleases which may be suitable include Cas9 isolated from: Staphylococcus aureus (SaCas9), Streptococcus thermophilus (StCas9), Neisseria meningitidis (NmCas9), Francisella novicida (FnCas9), and Campylobacter jejuni (CjCas9), and Streptococcus canis (ScCas9), as well as endonuclease variants or homologues of these naturally occurring Cas9 enzymes.

In some embodiments, the methods of the invention may be performed with an enzyme other than a Cas endonuclease. Suitably the methods of the invention may be performed with any enzyme which is capable of producing a targeted double strand break in DNA. For example, the methods of the invention may be performed using any nuclease or endonuclease, suitably restriction endonuclease.

The cell or population of cells may be prokaryotic, for example archaeal, or eukaryotic. Thus in some embodiments the cell or population of cells may be prokaryotic. In some embodiments the cell or population of cells may be bacterial, and in some embodiments the cell or population of cells may be archaeal. In some embodiments, the organism, cell or population of cells may be eukaryotic, for example animalia, fungi or plantae.

Suitably the organism, cell or population of cells may be derived from a mammal, bird, invertebrate, fish, reptile, amphibian. In some embodiments, the organism, cell or population of cells is mammalian. The organism or cell(s) may be mouse, rat, rabbit, sheep, goat, horse, cow, pig, dog, cat, primate, chicken, or human.

The population of cells may be obtained (or may have previously been obtained) from an organism, e.g. from the body of a mammal. Alternatively, the population may be derived by expanding in culture a cell or a plurality of cells obtained from an organism.

In some embodiments the cells are immune cells. Suitable immune cells may be: lymphocytes such as T-cells, B-cells, NK cells, or may be myeloid cells such as neutrophils, eosinophils, basophils, mast cells, dendritic cells, monocytes, or macrophages. In some embodiments, the cell(s) are T cells. Suitably the T-cells may be killer, helper or regulatory T-cells. In some embodiments, the cell(s) are CAR-T cells. Thus in some embodiments there are provided methods of generating genetically edited T-cells, comprising identifying a target sequence or guide RNA according to a method of the invention and then editing the genomes of a population of T-cells at the site identified by the target sequence or guide RNA. Preferably the genome of the population of T-cells will be edited by targeting a CRISPR-Cas endonuclease, for example Cas9, to the target sequence in the T-cell genome using a guide RNA selected according to a method of the invention.

In some embodiments, the cells are progenitor cells or stem cells. Suitable stem cells include primary stem cells, or immortalised stem cells. In one embodiment, the cells are induced pluripotent stem cells. Suitably the progenitor cells or stem cells are human.

In some embodiments, the organism, cell, or population of cells may be a modified organism, cell, or population of cells. In some embodiments, the organism, cell, or population of cells may be genetically modified. Suitably therefore the methods of the invention may be carried out on organism, cell, or population of cells that have already been modified, i.e. on transgenic organisms, cells, or populations of cells.

In some embodiments, methods comprising the step of obtaining the cells from the organism are excluded from the scope of the invention.

Thus, in some embodiments, the method is for editing the target sequence in each cell of a population of cells ex vivo. The method may be for editing a target gene in each cell of a population of cells ex vivo. In some alternative embodiments, the method is for editing the target sequence in vivo, for example for editing a target gene in vivo. Editing in vivo may be as part of a therapeutic method or, alternatively, editing in vivo may be as part of a non-therapeutic method. For example, editing may be carried out in a non-human eukaryotic cell in vivo in order to generate a tissue or organism for experimental use. Thus, preferred methods are methods of generating model organisms, for example mice or rat models.

In some embodiments, following introduction of the guide RNA molecule and the Cas endonuclease into the cells of the population, CRISPR-Cas editing of the target sequences occurs such that at least 30%, at least 40%, at least 50%, at least 60%, at least 70%, at least 80%, at least 90%, at least 95%, at least 98%, at least 99% or substantially all of the cells within the population have the same genotype (editing outcome).

In some embodiments, the cell is a zygote. In some embodiments, the zygote is non-human. Preferred methods of the invention do not encompass processes for modifying the germ line genetic identity of human beings. Suitably references herein to embryo or zygote may be a non-human embryo or zygote.

Thus, the invention may provide a method of producing a non-human, optionally mammalian, transgenic animal, the method comprising introducing a Cas endonuclease, preferably a Cas9 endonuclease, and a guide RNA molecule into an embryo, wherein the guide RNA molecule comprises a guide RNA sequence which, when used in CRISPR-Cas editing, results in (or is predicted to result in, e.g. by a computer model), a major editing outcome having a frequency which is at least 2-fold greater than the second most abundant editing outcome. Preferably the guide RNA is selected using a method of selecting a guide RNA according to the invention, as detailed above. Alternatively, the invention may provide a method of producing a non-human transgenic animal, the method comprising introducing the or each Cas endonuclease, preferably a Cas9 endonuclease, and the or each guide RNA molecule into an embryo, and performing the steps of the second aspect.

In one embodiment the invention may provide a method of producing a chimeric animal. Suitably the chimeric animal may be an interspecies chimera or an intraspecies chimera. Suitably such a method may comprise modifying a nucleic acid sequence in a cell or a population of cells derived from a first organism by carrying out a method of the invention, and implanting the cell or population of cells into a second organism. Suitably once implanted, the modified cell or population of cells may grow, and expand. Suitably the first organism may be a human and the second organism may be a different mammal, for example a pig. In some embodiments, the cell or population of cells may be an non-human embryo. In some embodiments, the cell or population of cells may be stem cells, pluripotent stem cells, or progenitor cells. Preferably such a method does not encompass processes to produce chimeras from germ cells or totipotent cells of humans and animals.

Optionally, the method further comprises transferring the embryo into a recipient female animal for gestation.

The Cas endonuclease and a guide RNA molecule may be introduced into the embryo at the one-cell stage (i.e. zygote). The zygote may be cultured to a later stage of development (e.g. the two-cell, four-cell or eight-cell stage) prior to transfer.

Thus, in some embodiments, the method of the fourth aspect of the invention is for editing a, or a plurality of nucleic acid sequences, for example a gene, in an embryo. The method may comprise introducing the or each guide RNA molecule and the Cas endonuclease into each cell of the embryo. The or each guide RNA molecule and the Cas endonuclease may be introduced into the cells of the embryo at the 2-cell, the 4-cell, or the 8-cell stage, or later.

In some embodiments, multiple embryos are transferred into a single recipient female. For example, at least 2, at least 3, at least 4, at least 5, at least 8, at least 10 or at least 15 embryos may be transferred into a recipient female. This may result in the birth of multiple live offspring. Advantageously, more than 25% of the offspring may be non-mosaic. In some embodiments at least 30%, at least 40%, at least 50%, at least 55%, at least 60%, at least 70%, at least 80%, at least 90% or 100% of the offspring are non-mosaic. By “non-mosaic” it will be understood that substantially all cells (in substantially all tissues) within an individual animal have the same genotype.

The present invention thus provides a method of reducing or eliminating mosaicism in transgenic animals in a single generation, without the need for subsequent breeding steps. Accordingly, the methods of the invention may be used to generate a non-mosaic transgenic animal.

The animal maybe a mammal. The animal may be a rodent, such as a mouse or a rat. Alternatively, the animal may be a rabbit, sheep, goat, horse, cow, pig, dog, cat, chicken or primate. The primate may be non-human.

Thus, in yet a further aspect, there is provided cells (e.g. embryos), cell populations and non-human organisms (e.g. transgenic animals) obtainable by the methods described herein.

Suitably such cells, cell populations and non-human organisms are modified cell populations and modified non-human organisms.

Embodiments of the invention will now be described by way of example and with reference to the accompanying figures in which:

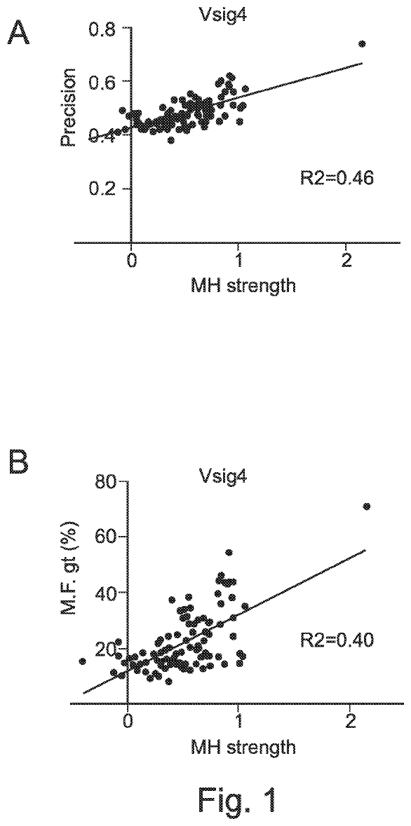

FIG. 1A is a graph showing microhomology strength plotted as a function of precision in double strand break (dsb) repair for the Vsig4 gene. Precision can be understood as the predictability of the repair outcome;

FIG. 1B is a graph showing microhomology strength plotted against most frequent genotype (M.F.gt) for the Vsig4 gene. High microhomology reduces the spectrum and complexity of repair results, giving rise to more consistent outcomes;

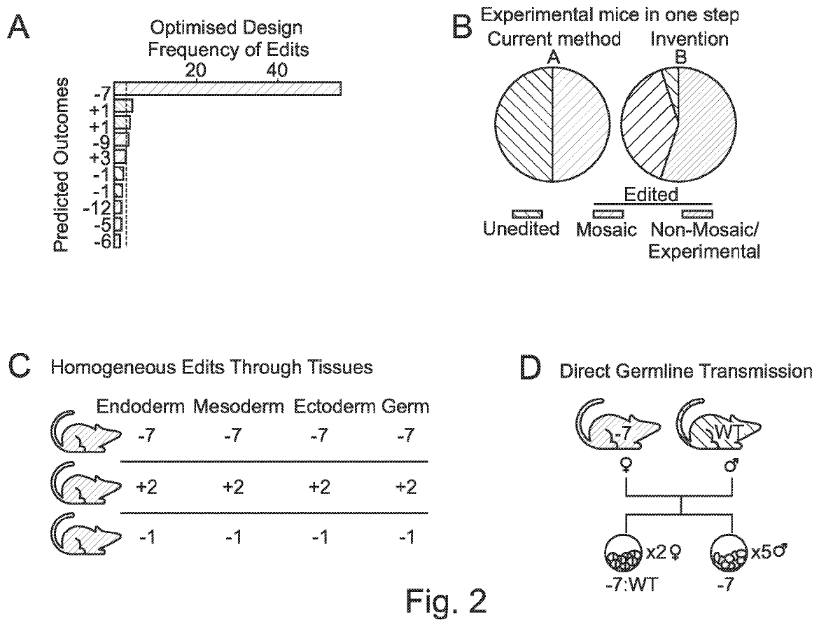

FIG. 2A is a graph showing the predicted editing outcomes of Vsig4 CRISPR design versus the frequency of each edit, a 7 bp deletion has by far the highest frequency;

FIG. 2B(A) is a chart showing the results of a representative example of creating a CRISPR murine model generated without attention to microhomology at the target site. Half of the pups born were unedited and half were mosaic;

FIG. 2B(B) is a chart showing the outcome of creation of a CRISPR murine model when knowledge of microhomology was applied. Less than half of mice born were unedited or mosaic. The majority (21/38) were non-mosaic and experiment-ready;

FIG. 2C shows the results of DNA sequence analysis of individual tissues in three representative genetically modified mice. The same insertions and deletions (7 bp deletion, 2 bp insertion, 1 bp deletion) were observed throughout different tissues that originated from disparate developmental lineages;

FIG. 2D shows representative data of direct germline transmission of edits. Oocytes from an edited female were fertilized by a wild type (WT) male and cultured to the blastocyst stage before lysis and sequence analysis. Blastocysts showed inheritance of the genetic modification in every case. As expected, inheritance followed a pattern characteristic of a sex-linked gene;

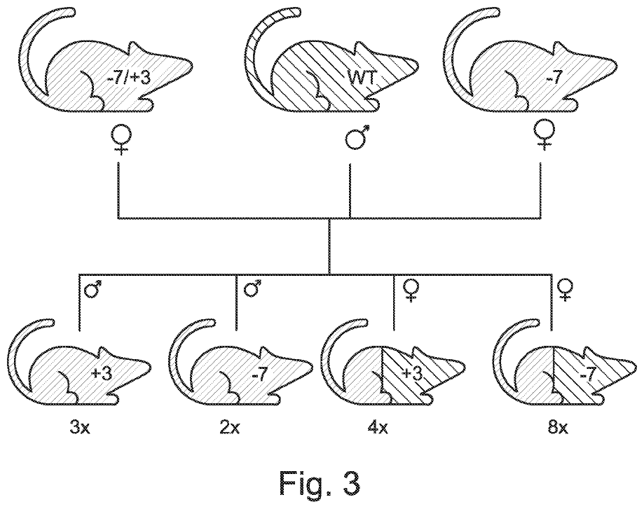

FIG. 3 depicts the results from breeding a trio of mice comprised of a wild-type male and two non-mosaic females with Vsig4 modifications, to assess germline transmission. The resultant pups all contained edits in a pattern consistent with an X-linked gene. All males showed complete editing while the females were heterozygous for the gene edit. Importantly, there were no entirely wild type mice or unexpected gene edits in the litter, indicating complete transmissibility of genome modification;

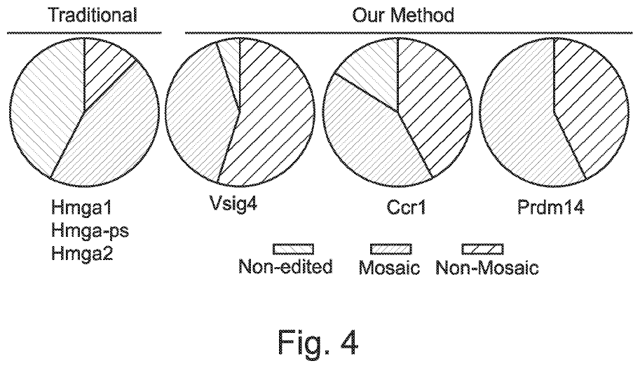

FIG. 4 shows that the method of the invention performed with respect to Vsig4 is repeatable and generalisable to other genes. The pie charts show aggregated data from performing the method of the invention exploiting local DNA sequence in genes Vsig4, Ccr1, and Prdm14, compared with a chart showing the data for traditional methods that ignore local DNA sequence features in genes Hmga1, HMga1-ps, and Hmga2;

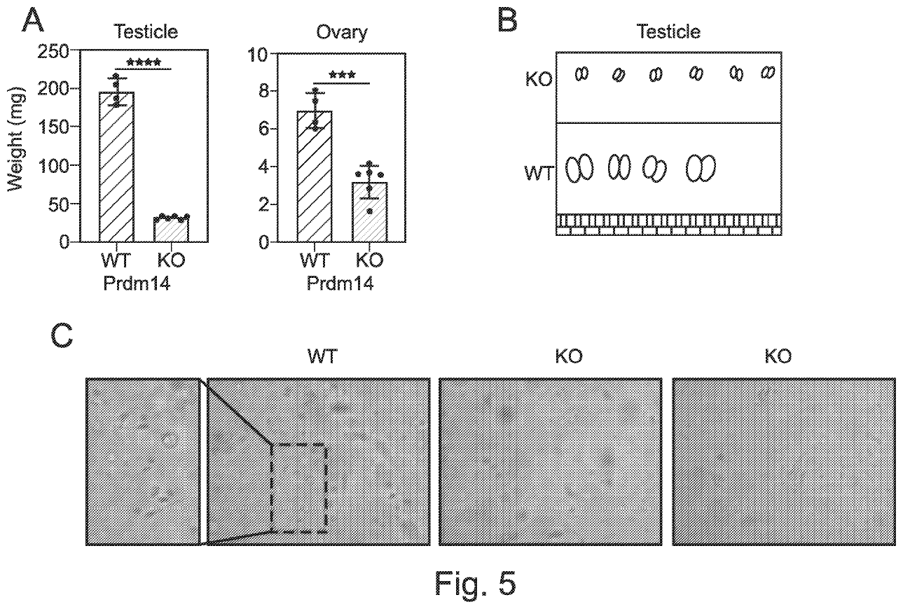

FIG. 5 shows the functional analysis of a Prdm14 knock out for which there is a phenotypic effect (A) Testes and ovarian tissue from genotyped Prdm14−/− mice were excised and weighed. Statistical analyses were performed using a Students T-test, yielding P=9.4×10-9 (Testicle) and P=2.2×10-4 (Ovary). (B) Visualisation of testicular tissue. (C) Microscopic observation of spermatids (red asterisk) in wt but not in Prdm14−/− males;

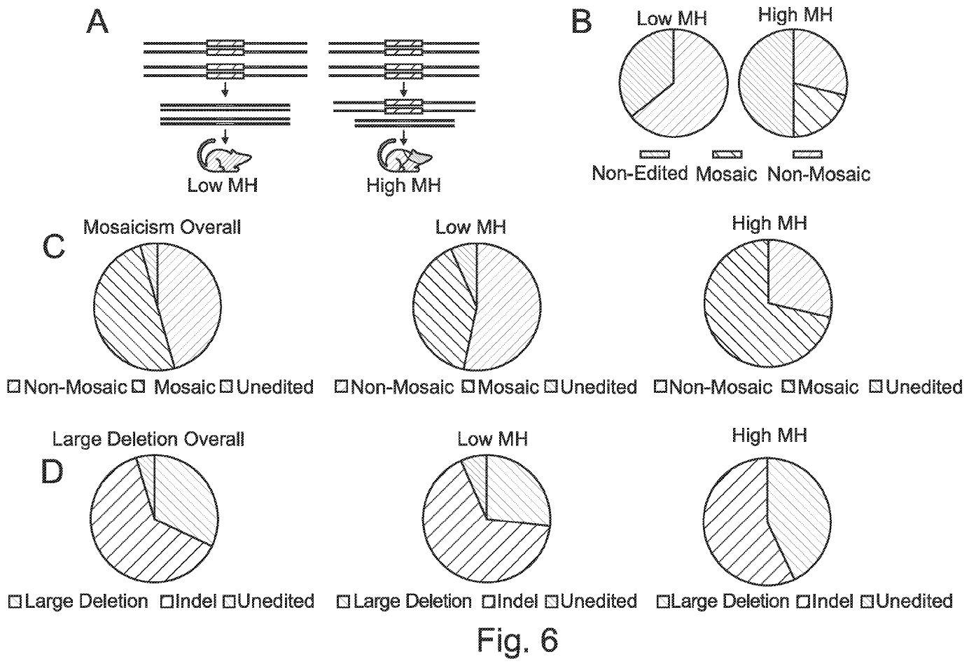

FIG. 6 shows the ability to use the method of the invention to enhance large deletions by analysing DNA microhomology, (A) Homozygous large deletions can be enhanced by targeting regions of low local microhomology, giving rise to non-mosaic animals. (B) Preliminary data using the gene Ddx3y shows that regions of low microhomology more frequently result in biallelic large deletions compared to targeting regions of high microhomology, (C) and (D) Pie charts showing the raw data for performing the same methodology in the gene Gata1. In (C) from left to right: Pie chart of overall mosaicism for Gata1 model, ‘non-mosaic’ is when a single editing outcome was present for both editing sites. These editing outcomes include indels and the desired deletion; Pie chart for mosaicism for Gata1 model when only looking at a low microhomology guide RNA pair. These editing outcomes include indels and the desired deletion; Pie chart for mosaicism for Gata1 model when only looking at a high microhomology guide RNA pair. These editing outcomes include indels and the desired deletion. In (D) from left to right: Pie chart of large deletions present in all Gata1 edited mice. Large deletions, indels, and unedited mice are compared (Not indicative of mosaicism); Pie chart of large deletions present in Gata1 mice generated using a low microhomology guide RNA pair; Pie chart of large deletions present in Gata1 mice generated using a high microhomology guide RNA pair;

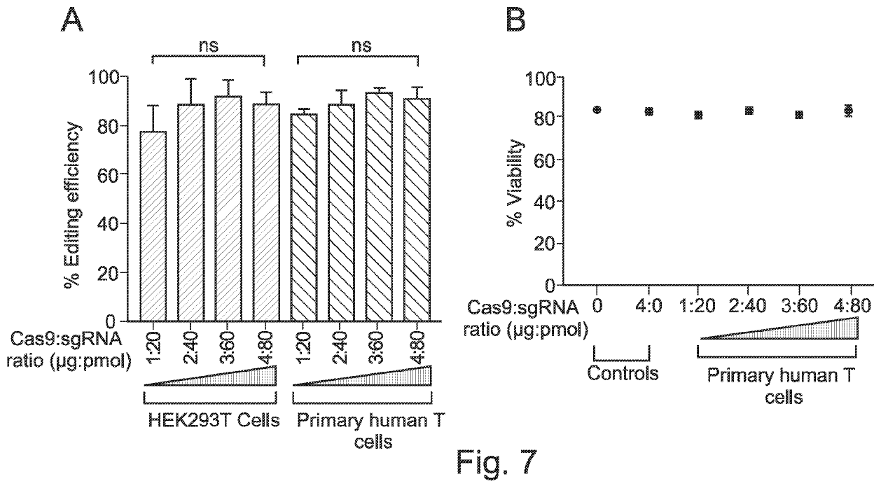

FIG. 7 shows efficient Cas9 editing of primary human T cells without loss of viability. HEK293T and primary human T cells were edited with guides designed against CTLA4 using the method of the invention (A) Editing efficiency of increasing amounts of Cas9:sgRNA targeting CTLA4 in HEK293T cells and primary human T cells (n=3, technical replicates, error bars=SD, ns=nonsignificant). Efficiency was measured by determining the proportion of cells that have indels that led to a frameshift in the protein-coding region compared to the unedited control. (B) T cell viability recorded 3 days post edit. The % viability was calculated as the percentage of live cells/mL to total cells/m L;

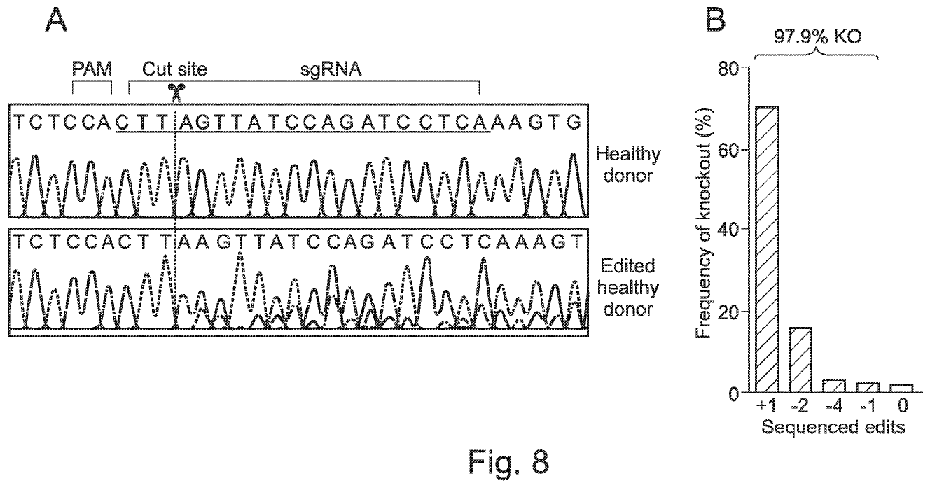

FIG. 8 shows (A) Sequencing traces of healthy donor T cells edited with an sgRNA targeting CTLA4. The sgRNA target sequence is underlined in black. The contribution a particular edit within the pool of edited cells is shown. (B) The distribution of editing outcomes within the pool is seen. Greater than 70% of the edits are pure indicating that the level of mosaicism has been reduced to below 30%;

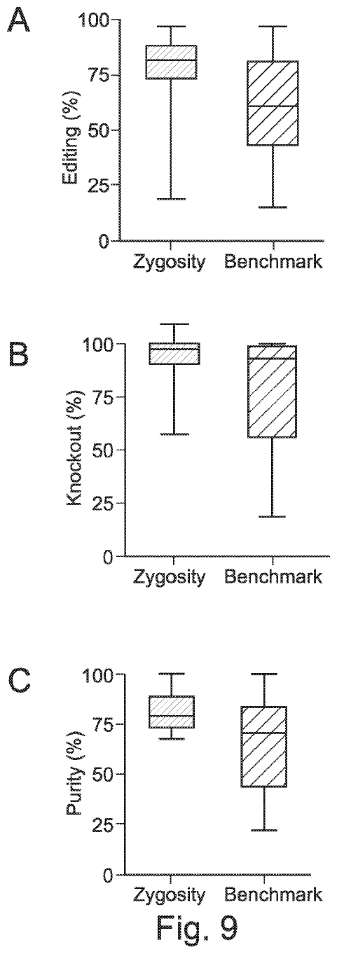

FIG. 9 shows (A) Comparison of editing efficiency between gRNAs designed using the method of the invention (Zygosity) and Sanger designed (benchmark) gRNAs to CTLA4, PD-1, LAG-3, PTPN2, DGK & HAVCR2 (also known as TIM3) in primary T-Cells. gRNAs designed according to the invention were more efficient as editing primary T-cells across 6 genes (P<0.005) (B) Comparison of knockout efficiency between gRNAs designed using the method of the invention (Zygosity) and Sanger designed (benchmark) gRNAs to CTLA4, PD-1, LAG-3, PTPN2, DGK & HAVCR2 in primary T-Cells. gRNAs designed using the method of the invention knocked out a higher portion of genes in primary T-cells when compared to Sanger designed guides (P<0.006) (C) Comparison of the extent of mosaicism between gRNAs designed using the method of the invention (Zygosity) and Sanger designed (benchmark) gRNAs to CTLA4, PD-1, LAG-3, PTPN2, DGK & HAVCR2 in primary T-Cells. gRNAs designed using the method of the invention resulted in decreased mosaicism (indicated by increased Purity (%)) in primary T-cells when compared to Sanger designed guides (P<0.006);

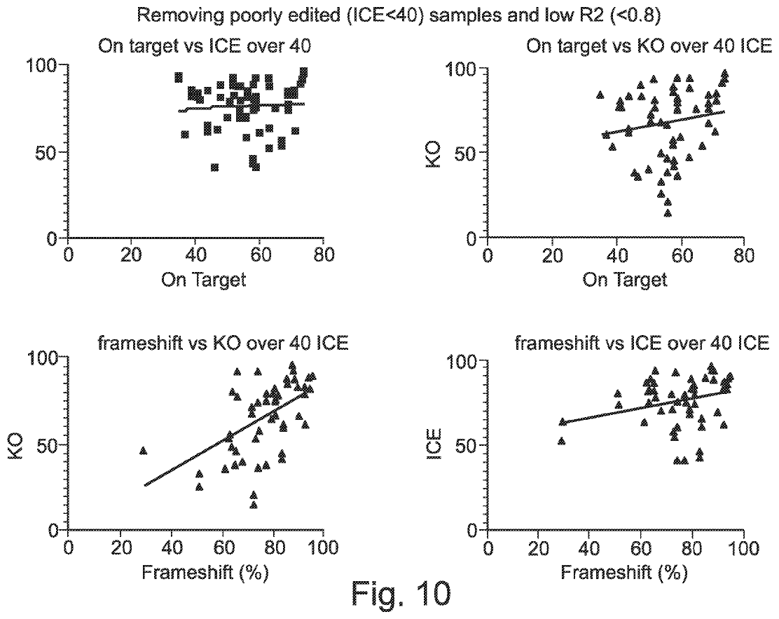

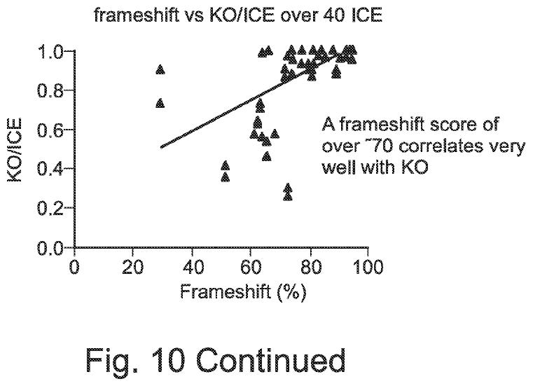

FIG. 10 shows the correlation between various computationally derived metrics describing guide performance (on-target, predicted frameshift frequency) with editing outcome derived from Sanger sequencing of edits. The data show that the best predictor of gene knockout efficiency is the frameshift metric as used in the present invention;

CRISPR editing using the guide RNA sequences selected in accordance with the methods of the invention may be carried out using the following protocols:

### EXAMPLE PROTOCOL FOR EDITING EMBRYOS

1. Order guide RNA sequences as synthetic modified single guide RNAs (sgRNAs) (e.g. from Merck);
2. Resuspend sgRNAs in water;
3. Have prepared in vitro fertilized mouse zygotes ready to electroporate at 3 hours post-insemination;
4. Complex 4.5 μg of sgRNA with 20 μg Cas9 protein (TrueCut V2, Invitrogen) in 60 μL Opti-MEM (Thermofisher) at room temperature for 20 minutes;
5. Transfer 50 μL of the complex solution to the CUY520P5 electrode of a NEPA21 electroporator;
6. Adjust the volume until the impedance is within the range of 0.48-0.52 kW;
7. Move Opti-MEM washed zygotes to the electroporation chamber;
8. Check impedance again;
9. Electroporate using parameters outlined in Table 1;
10. Remove zygotes from the electroporation chamber and place in KSOM solution for 30 minutes;
11. Wash zygotes with KSOM solution three times;
12. Return zygotes to fresh KSOM solution and culture until at least the two-cell stage, in some cases to the blastocyst stage;
13. Transfer to pseudo-pregnant recipient female mice.

TABLE 1
List of parameters for NEPA21 electroporation

| | Set parameters | | | | | | | | | | |
|---|---|---|---|---|---|---|---|---|---|---|---|
| | Poring pulse | | | | | Transfer pulse | | | | | |
| | | | | D. | | | | | | D. | |
| | Length | Interval | | Rate | | | Length | Interval | | Rate | |
| V | (ms) | (ms) | No. | (%) | Polarity | V | (ms) | (ms) | No. | (%) | Polarity |
| 200 | 2 | 50 | 4 | 10 | + | 20 | 50 | 50 | 5 | 40 | +/− |

### EXAMPLE PROTOCOL FOR EDITING CELLS

1. Order guide RNA sequences as synthetic modified single guide RNAs (sgRNAs) (e.g. from Merck);
2. Resuspend sgRNAs in water;
3. Complex 80 pmol of sgRNA and 4 ug of Cas9 protein (TrueCut V2, Invitrogen);
4. Harvest cells (˜400,000), pellet, wash with 1×PBS, and resuspend in 20 μL of appropriate electroporation buffer.
a. Cell count and volumes are appropriate for use with Lonza's 16-well cassette. Scale up and follow kit procedure when using different electroporation vessel;
b. Use Amaxa 4D nucleofector and purchase electroporation vessels and buffers through Lonza. Different buffers are optimized for different use with cell types;
c. Once cells have been mixed with electroporation buffer, complete electroporation as soon as possible;
5. Mix cells with complexed sgRNA and Cas9 protein and transfer to electroporation vessel;
6. Electroporate according to cell type specific program recommended by Lonza;
7. Add 80 μL of cell media to electroporation cassette and place in incubator for 10 minutes for cells to recover;
8. Put cells into 12 well plate that contains 1 mL of pre-warmed media;
9. Culture as desired.

### EXAMPLE 1

Once thought random, the repair of the double stranded DNA break (DSB) induced by Cas9 cleavage of its target sequence has been recently shown to be non-random. Short areas of repetitive DNA sequence (microhomology) around the Cas9 cut site play a major role in how the DSB is repaired. As show in FIG. 1, there is a direct relationship between local microhomology and non-random repair, or precision, of a target sequence (FIG. 1A). The direct relationship allows us to understand and predict editing outcome de novo. DNA sequences with low microhomology repair to give a wide spectrum of editing outcomes in a manner that is difficult to predict. Higher microhomology correlates with a decreased spectrum of repair outcomes (FIG. 1B). Further, editing events may evolve over time through re-cutting of the target sequence and lead to mosaicism. The present inventors hypothesised that the relationship between double strand break repair and microhomology may be exploited in order to bias editing outcomes and reduce or eliminate mosaicism.

In embryos, by leveraging the linear relationship between microhomology and both precision and consistency, it was surprisingly found that it is possible to constrain the spectrum of edits to one major outcome. Restricting the editing spectrum nullifies re-cutting based mosaicism by biasing editing to produce a single genotype. The combination of these two approaches allows us to produce non-mosaic, experiment-ready mice in one step.

### Materials and Methods

### CRISPR Guide Design

SpCas9 single guide RNAs for Vsig4 (ENSMUSG00000044206) were selected in accordance with the following protocol:

1. The primary transcript was identified using a publicly available genomics tool, enseumble.org;
2. All possible guide RNAs that target the coding sequence of Vsig4 were identified using the publicly available software FOREcasT, InDelphi or Lindel. Other suitable software includes UCSC Genome Browser, Deskgen.com, and CRISPOR;
3. Guide RNA sequences which targeted the second 50% of the gene were filtered out;
4. Guide RNA sequences were analysed using Lindel using the metric “Most Frequent Genotype (MF gt)” and the fold change between the most abundant editing outcome and the second most abundant editing outcome was calculated for each guide RNA sequence. The top 10 ranking guide sequences were selected;
5. Guide RNA sequences were ranked using Lindel using the metric “frameshift %”. Guides for which the major editing outcome was not a multiple of three were selected;
6. Guide RNA sequences were assigned an off-target score using the webtool Deskgen. Other suitable tools include UCSC Genome Browser and CRISPOR. The algorithm used by Deskgen and most other tools is that of Hsu et al., (Nature Biotechnology volume 31, pages 827-832(2013)). In the webtool Deskgen the scores range from 0 (many off targets) to 100 (no off targets). Guides with a score of less than 70 were filtered out;
7. Guide RNA sequences with undesirable on-target profiles were filtered out using Deskgen. which assigns a score of 0-100 based on the metric described by Doench et al., (Nature Biotechnology volume 34, pages 184-191(2016)). Guides having scores of more than 35 (which have been found to work well in vitro and in vivo) were selected;
8. The top three ranked guide RNA sequences were tested by carrying out CRISPR gene editing in mouse ES cells. Synthetic phosphorothioate-modified sgRNAs for Vsig4 were purchased from Merck (UK). Following editing, genomic DNA was then extracted and sequenced across the edited region using standard techniques to determine the editing percentages and distribution of edits. The information was analysed using the ICE v2 CRISPR Analysis Tool (Synthego).
9. The guide RNA sequence which was found to result in the least mosaicism was then used to generate transgenic mice.

### Super-Ovulation

Female C57Bl6/J (Charles River, UK) at 10-14 weeks old were super-ovulated by intraperitoneal (ip) administration of 7.5 IU of Pregnant Mare Serum Gonadotropin (National Veterinary Services, 859448), followed by ip injection of 7.5 IU of Human Chorionic Gonadotropin (hCG) (National Veterinary Services, 804745) 48 hours later. Oocytes were harvested from the super-ovulated females 14-16 hours post-hCG injection.

### In Vitro Fertilization (IVF)

Human Tubal Fluid (HTF) medium was prepared in water using the reagents listed in Table 2, and sterile filtered (0.2 μm).

TABLE 2
HTF medium components

| | Final | | Merck Catalogue |
|---|---|---|---|
| Reagent | Concentration | | Number |
| Sodium Chloride | 101.6 | mM | S5886 |
| Potassium Chloride | 4.69 | mM | P5405 |
| Magnesium Sulphate Heptahydrate | 0.2 | mM | M7774 |
| Monopotassium Phosphate | 0.37 | mM | P5655 |
| Calcium Chloride Dihydrate | 2.04 | mM | C7902 |
| Sodium bicarbonate | 25 | mM | S5761 |
| Glucose | 2.78 | mM | G6152 |
| Sodium pyruvate | 0.33 | mM | P4562 |
| Sodium lactate 60% syrup | 21.4 | mM | L7900 |
| Penicillin-G | 0.075 | g/L | P4687 |
| Streptomycin Sulphate | 0.05 | g/L | S1277 |
| Phenol Red (5%) | 0.2 | mL | P0290 |
| Bovine Serum Albumin (Embryo | 4 | g/L | |
| Tested) | | | |

10 μL of cryopreserved sperm was added to 490 μL HTF medium containing 1.25 mM reduced L-glutathione (rGSH) (Merck, G-4251) in a 4 well tissue culture dish and pre-incubated for 45 min. Oocytes from super-ovulated females were harvested and transferred into the media containing the thawed sperm and incubated for 2 hours. Zygotes visibly showing a second polar body were collected and washed three times in pre-prepared KSOM solution (KSOM medium (Merck Millipore, MR-107-D) and 3 mg/mL bovine serum albumin (BSA) (Sigma-Aldrich, A-3311)). Zygotes were cultured in 1 mL KSOM solution until electroporation.

### Embryo Electroporation

sgRNA target sequence as shown below (5′-3′):

Vsig4 guide:
(SEQ ID No. 1)
ATGATCCCCTGAGAGGCTAC

4.5 ug of sgRNA was complexed with 20 μg TrueCut Cas9 protein v2 (Invitrogen) in 60 μL Opti-MEM (ThermoFisher) at room temperature for 20 min. 50 μL of this solution was transferred to the CUY520P5 electrode of a NEPA21 electroporator (Nepa Gene) and the volume was adjusted until the impedance was within range of 0.48-0.52 kΩ Opti-MEM washed zygotes were added to the electroporation chamber and the impedance was assessed again to ensure it fell within the range. The parameters for electroporation using the NEPA21 are shown in Table 1, above.

Zygotes were removed from the electroporation chamber and placed into KSOM solution for 30 min. The zygotes were washed with KSOM solution three times, returned to fresh KSOM solution and cultured until they reached the two-cell stage.

### Embryo Transfer

Female CD1 mice (Charles River, UK) were mated with vasectomised males. Two-cell stage embryos were surgically transferred into the oviduct of pseudo-pregnant recipient females, 10 embryos per oviduct, 20 embryos per female.

### Genotyping and Deconvolution of Editing Outcome

Zygotes were cultured in KSOM solution to blastocyst stage where the zona pellucida was removed using Tyrode's Solution (Sigma-Aldrich, T-1788) and the samples lysed in extraction reagent (Quanta, 84158). DNA was extracted from tissue (ear biopsy, lung, heart, liver, or testicle) using E.Z.N.A. Tissue DNA Kit (Omega, D3396-01). PCR amplification of the region surrounding Vsig4 sgRNA target sites was performed using the following primers (5′-3′):

Vsig4-F:
(SEQ ID No. 2)
CCTAACTCTCACATAATATT
Vsig4-R:
(SEQ ID No. 3)
ATTACAGAGAACCTATGTAC

PCR amplification from tissue samples was performed using Q5 High Fidelity DNA polymerase and master mix (NEB). Vsig4 cycling conditions: 98° C. for 30 seconds, 35 cycles of (98° C. for 10 seconds, 50° C. for 30 seconds, and 72° C. for 45 seconds), and 72° C. for 5 min.

PCR amplification from blastocyst samples was performed using Phusion polymerase and HF buffer (NEB). Vsig4 cycling conditions: 98° C. for 3 min, 35 cycles of (98° C. for 30 seconds, 50° C. for 30 seconds, and 72° C. for 45 seconds), and 72° C. for 5 minutes.

PCR samples were cleaned up using the QIAquick PCR Purification kit (Qiagen) and Sanger sequenced (Eurofins Genomics). Sequence deconvolution of the sanger traces were determined using the Inference of CRISPR Edits (ICE) tool (Sythego).

### Results

By constraining CRISPR activity to regions of high microhomology within the X-linked gene, V-set and immunoglobulin containing 4 (Vsig4), it was demonstrated that mosaicism can be eliminated. Single guide RNA (sgRNA) was pre-complexed with Streptococcus pyogenes Cas9 (SpCas9) protein, and the complex was electroporated into in vitro fertilized zygotes at three hours post insemination. Zygotes were transferred into pseudopregnant female recipient mice for live birth. Pups were genotyped from ear biopsies by PCR amplification around the targeted cut site, Sanger sequencing, and deconvolution to identify mosaicism. Over half (21/38, 55%) of pups were non-mosaic. Comparatively, other studies conducted in the lab have resulted in only mosaic or non-edited animals (FIG. 2A). The extent of editing in disparate tissues was determined by extracting DNA from organs that derived from different developmental lineages. Tissue was extracted from the liver, epidermis, heart, and testicles which originate from the endoderm, ectoderm, mesoderm, and germ cells respectively. The entire organ was digested and the DNA was extracted and analysed. All animals analysed (N=7) had identical editing outcomes throughout the tissues, indicating that mosaicism was efficiently eradicated across all developmental linages (FIG. 2B(B)).

Creation of non-mosaic founder animals who transmit their induced genetic modifications to the next generation is critical to rapidly create a breeding. To test this, oocytes from a non-mosaic female that contained a 7 base pair (bp) deletion were in vitro fertilized with sperm from a wild type male and the resultant zygotes were cultured until the blastocyst stage. Seven blastocysts were individually collected, lysed, and analysed for presence of the genetic modification. Two of the seven blastocysts had a genotype of 50% wild type and 50% 7 bp deletion, while the remaining five contained only the 7 bp deletion (FIG. 2C). The inheritance pattern is characteristic of a sex-linked gene, like Vsig4. Germline transmission was further characterized by setting up a breeding trio comprised of a wild type male and two edited females (FIG. 3). All examined animals were able to pass their genetic modification onto the next generation.

### EXAMPLE 2

The same methodology as used in the Vsig4 gene described above was repeated in other genes: Ccr1 and Prdm14. SpCas9 single guide RNAs for Ccr1 and Prdm14 were designed as above. By extending the method of the invention into other models, it is demonstrated that the methodology is generalisable. In each instance where the method was tested, non-mosaic, experimental cohorts were produced.

Importantly, the Ccr1 experiment was performed on a complex genetic background (Trp53R172H/Pdx1-Cre), demonstrating that the approach works on pre-established disease models, not only in wild type genetic contexts. Prdm14 plays a key role in the specification of the primordial germ cell (PGC); mutation results in sterility. Thus, Prdm14−/− lines cannot be established and bred, however a cohort of non-mosaic Prdm14−/− lines can be produced on demand using the method of the invention. Results for these experiments are shown in FIG. 4. Results for the Prdm14−/− with respect to phenotype are shown in FIG. 5.

Ccr1 sgRNA:
(SEQ ID NO 4)
CTCTCTGGGTTTTATTACCT
Prdm14 sgRNA:
(SEQ ID NO 5)
GGTCAATGCCAGCGAAGTGA

| Gene | Primer-Forward | Primer-Reverse |
|---|---|---|
| Ccr1 | ATGGAGATTTCAG | CCTTCCTTCTCAC |
| | ATTTCACAGAA | TGGGTCTT |
| | (SEQ ID NO 6) | (SEQ ID NO 7) |
| Prdm | TAAATCCTCTCT | TTTCCTGTAGCA |
| 14 | AGGGACTG | TGCTTTTA |
| | (SEQ ID NO 8) | (SEQ ID NO 9) |

### EXAMPLE 3

Furthermore, the inventors have investigated the use of the method of the invention not only to predict which guide RNA to use to enhance a single editing outcome but also to predict which pairs of guide RNAs should be used to achieve a large deletion.

It may be desirable to generate models that harbour large genomic deletions, either to explore functions of the deleted region, or as an alternative approach to generate a gene knockout. The inventors believe that DNA regions which repair into many editing outcomes (mosaic) incur a delay during the repair process as the cell searches for a compatible (local) sequence to repair the insult when compared to DNA regions which repair into a single, dominant editing outcome (non-mosaic). By using two guide RNAs flanking a desired genomic region that exploit this proposed repair delay it should enhance the efficiency of large deletion events.

The Y-linked spermatogenesis regulator, Ddx3y, was targeted for knockout. CRISPR designs were constrained to regions surrounding a critical exon of Ddx3y, such that removal of the exon would move the coding sequence out of frame. Pairs of guide RNAs were designed that target regions of either high or low microhomology that were predicted to result in few or many editing outcomes respectively using the guide design protocol above (FIG. 6A). To select the gRNAs which target regions of high microhomology, the same method as above was used. To select guide RNAs which target regions of low microhomology, step 4 in the design method above comprised selecting the bottom 10 gRNAs, step 5 is omitted, step 8 comprised selecting the bottom 3 gRNAs, and the final step comprised selecting the gRNA which was found to result in the highest mosaicism. This was done for both 5′ flank and the 3′ flank regions surrounding the DNA sequence to be deleted.

Zygotes were edited in vitro as explained above, using both a gRNA that targets the 3′ flank and a gRNA that targets the 5′ flank of the intervening DNA sequence to be deleted, and analysed by PCR as explained above and sequencing individual blastocysts. The data show that both conditions generated deletion events, however more were generated in the low microhomology group (63% vs 28%) (FIG. 6B). These data show that low microhomology flanking pairs of guide RNAs enhance the excision of intervening DNA sequence.

The concept of using pairs of gRNAs which target regions of low microhomology to enhance deletions was further investigated in the context of the gene Gata1. FIGS. 6C and 6D show the results. FIG. 6D in particular shows that using pairs of gRNAs targeted to regions of low microhomology generates a greater proportion of large deletions than targeting pairs of gRNAs to high microhomology regions.

DDX3y_5′_HMH sgRNA:
(SEQ ID NO 10)
TCCAGTGTCTATCACTGTAC
DDX3y_3′_HMH sgRNA:
(SEQ ID NO 11)
TAGTAAATTCTTAGGTAAGT
DDX3y_5′_LMH sgRNA:
(SEQ ID NO 12)
CCCAGTACAGTGATAGACAC
DDX3y_3′_LMH sgRNA:
(SEQ ID NO 13)
AATCTTAACTTAGCAAAGTC
Gata1_5′_HMH sgRNA:
(SEQ ID NO 14)
GCCGCAGTAACAGGCTGTCT
Gata1_3′_HMH sgRNA:
(SEQ ID NO 15)
ACGCCAGCTCTGGCCTGCTC
Gata1_5′_LMH sgRNA:
(SEQ ID NO 16)
CTGTCTTGGGGCTGGGGGGC
Gata1_3′_LMH sgRNA:
(SEQ ID NO 17)
CCAGAGCTGGCGTAAGCCCC

| Gene | Primer-Forward | Primer-Reverse |
|---|---|---|
| Gata1 | TGTCCCTGCTGCT | GTTGGACCTGTAT |
| | TTCTGTC | GCGCGTG |
| | (SEQ ID NO 18) | (SEQ ID NO 19) |
| DDX3y | TACCAAGCCACA | AATCCGGGCCACA |
| | TTTGTAGCTC | GCTTCTTGT (SEQ ID |
| | CC (SEQ ID NO 20) | NO 21) |

### EXAMPLE 4

CRISPR-Cas9 editing of CAR-T cells suffers from generalised inefficiency/toxicity and mosaicism. In this context, both these factors serve to limit the therapeutic potential and safety profile of these next generation therapies. The method of the invention was further used to generate a SpCas9 single guide RNA to an intron in CTLA4 and tested in HEK293T and in primary human T cells.

CTLA4_intron sgRNA:
(SEQ ID NO 22)
TGAGGATCTGGATAACTAAG

| Gene | Primer-Forward | Primer-Reverse |
|---|---|---|
| CTLA4_intr | CTCTGTATTCCAGGGCC | CAGTGAAATGGCTT |
| on | AGC (SEQ ID NO 23) | TGCTCA |
| | | (SEQ ID NO 24) |

### Method to Stimulate PBMC Cells

Anti-CD3 antibody (Biolegend) was diluted to a final concentration of 5 μg/mL in sterile PBS and 50 μL per well was added to 3×96 well plates. Incubate plates at 37° C. for 2 hours. Wash each plate 3× with 200 μL PBS. Revive PBMC cells (Cambridge Bioscience) in 7 mL of warmed media (RPMI glutamax 21875-034, 10% HI-FBS, 1.75 μL BME). Centrifuge 3×5 min at 425 g. Resuspend final pellet in 10 mL media and count cells. Create cell suspension that contains cells at a concentration of 60,000 cells/200 μL, anti-CD28 antibody (Biolegend) to a final concentration of 5 μg/mL, and IL2 (Biolegend) to a final concentration of 20 ng/mL. Dispense cell suspension into prepared 96 well plates—60,000 cells/well and 200 μL/well. Leave in incubator 72 hours.

### Method for Electroporation of Stimulated Cells

Count cells and record viability. Only proceed with electroporation when the viability is over 65%. Complex 3 μg of TrueCut Cas9 protein v2 (Invitrogen) with 60 pmol of synthetic guide RNA (Synthego) at room temperature for 20 minutes. Pellet cells, wash with PBS, and pellet again. Resuspend cells in P3+ buffer (Lonza) at 200,000 cells per sample, mix with the Cas9/guide RNA complex and add 20 μL of this to the electroporation cuvette. Electroporate using program EO 115, incubate at 37° C. for 10 minutes and transfer to pre-warmed 24 well plates that contain a solution of 1 mL media with 20 ng/mL IL2 per well. Leave cells for 72 hours in the incubator before harvesting for analysis.

The inventors found that the generated guide RNA was highly efficient at gene editing over a broad concentration range, and across two cell types. The generated guide RNA had 90% gene editing efficiency of primary, patient-derived T cells levels comparable to the ubiquitous HEK293T cancer cell line (FIG. 7A); and cellular viability was maintained across the concentration range (FIG. 7B). Furthermore, the guide RNA also reduced mosaicism in primary T-cells as the vast majority of editing (70%) was a +1 bp insertion, the level of mosaicism was reduced to below 30% (FIGS. 8A and 8B).

Further guide RNAs were designed using the method of the invention for the genes CTLA4 (as above), PD-1(PDCD1), LAG-3, PTPN2, DGK & HAVCR2 in primary T-Cells, and their editing efficiency, knockout efficiency and purity were compared to gRNAs designed by a prior method; the Sanger (FIGS. 9A, B and C) method described in Tzelepis et al. Cell Reports, Volume 17, Issue 4, 18 Oct. 2016. The gRNAs designed by the method of the invention were more effective.

TABLE of
sgRNAs designed using either the method of
the invention or the prior Sanger method:

| | sgRNAs | | | | |
|---|---|---|---|---|---|
| | designed | | | sgRNAs | |
| | using the | | | designed | |
| | method of | SEQ | | using a | SEQ |
| | the | ID | | prior | ID |
| Gene | invention | NO | Gene | method | NO |
| CTLA4_1 | CATAAAGCC | 25 | CTLA4_6 | TCCATGCTAG | 55 |
| | ATGGCTTGC | | | CAATGCACG | |
| | CT | | | | |
| CTLA4_2 | TGAACCTGG | 26 | CTLA4_7 | CACAAAGCTG | 56 |
| | CTACCAGGA | | | GCGATGCCT | |
| | CC | | | | |
| CTLA4_3 | CTCAGCTGA | 27 | CTLA4_8 | CTGCCGAAGC | 57 |
| | ACCTGGCTA | | | ACTGTCACC | |
| | CC | | | | |
| CTLA4_4 | AGGGCCAG | 28 | CTLA4_9 | TGTGCGGCAA | 58 |
| | GTCCTGGTA | | | CCTACATGA | |
| | GCC | | | | |
| CTLA4_5 | CCTTGGATTT | 29 | CTLA4_10 | TTCACTTGATT | 59 |
| | CAGCGGCAC | | | TCCACTGG | |
| | A | | | | |
| PTPN2_11 | CTCTTCTATG | 30 | PTPN2_16 | GTGGATCACC | 60 |
| | TCAACTAAA | | | GCAGGCCCA | |
| | C | | | | |
| PTPN2_12 | CATGCCCAC | 31 | PTPN2_17 | GGGACTCCAA | 61 |
| | CACCATCGA | | | AATCTGGCC | |
| | GC | | | | |
| PTPN2_13 | CTCTTCGAA | 32 | PTPN2_18 | CGCATTGTGG | 62 |
| | CTCCCGCTC | | | AGAAAGAAT | |
| | GA | | | | |
| PTPN2_14 | GTTCAGCAT | 33 | PTPN2_19 | AGTTTAGTTG | 63 |
| | GACAACTGC | | | ACATAGAAG | |
| | TT | | | | |
| PTPN2_15 | TTGACATAG | 34 | PTPN2_20 | CATGACTATC | 64 |
| | AAGAGGCAC | | | CTCATAGAG | |
| | AA | | | | |
| DGKB_21 | TCTCTGGAG | 35 | DGKB_26 | ACATAGGTCT | 65 |
| | GAATGGATT | | | TGATGCAAG | |
| | CA | | | | |
| DGKB_22 | CTGGAGGAA | 36 | DGKB_27 | TCGAGCCACA | 66 |
| | TGGATTCAA | | | CAGCGCTCA | |
| | GG | | | | |
| DGKB_23 | GGTAAAATA | 37 | DGKB_28 | GAACATGCTG | 67 |
| | TGGTCCTTC | | | ATTGGCGTG | |
| | AA | | | | |
| DGKB_24 | ATGTGACTG | 38 | DGKB_29 | CGTCCCATGC | 68 |
| | TGGACCTTT | | | AGAACGTGA | |
| | GA | | | | |
| DGKB_25 | GGCACTTAT | 39 | DGKB_30 | TCGCCTTTAT | 69 |
| | CACACTTGG | | | GACACGGAT | |
| | TT | | | | |
| Lag3_31 | CGCCGGCGA | 40 | Lag3_36 | GCTCATCCAG | 70 |
| | GTACCGCGC | | | CTGGACGCG | |
| | CG | | | | |
| Lag3_32 | GGCTGAGGT | 41 | Lag3_37 | GTCCCGCCCC | 71 |
| | CCCGGTGGT | | | ACATACTCG | |
| | GT | | | | |
| Lag3_33 | AGGAGGGC | 42 | Lag3_38 | TGCATTGGTT | 72 |
| | GCCGCCGGG | | | CCGGAACCG | |
| | TGA | | | | |
| Lag3_34 | CGCTATGGC | 43 | Lag3_39 | ATGGGGGGA | 73 |
| | TGCGCCCAG | | | CTCCCGGACA | |
| | CC | | | | |
| Lag3_35 | CCCTGAGGT | 44 | Lag3_40 | GAGGAAGCTT | 74 |
| | GCACCGCGG | | | TCCGCTAAG | |
| | CG | | | | |
| HAVCR2_41 | AATGTGACT | 45 | HAVCR2_46 | CTCTCTGCCG | 75 |
| | CTAGCAGAC | | | AGTCGGTGC | |
| | AG | | | | |
| HAVCR2_42 | TGTGTTTGA | 46 | HAVCR2_47 | ATGTGACTCT | 76 |
| | ATGTGGCAA | | | AGCAGACAG | |
| | CG | | | | |
| HAVCR2_43 | TCTCTGCCG | 47 | HAVCR2_48 | TAAATGGGGA | 77 |
| | AGTCGGTGC | | | TTTCCGCAA | |
| | AG | | | | |
| HAVCR2_44 | GGTGTAGAA | 48 | HAVCR2_49 | GTGTTTGAAT | 78 |
| | GCAGGGCA | | | GTGGCAACG | |
| | GAT | | | | |
| HAVCR2_45 | AGAAGTGGA | 49 | HAVCR2_50 | ACGGGCACG | 79 |
| | ATACAGAGC | | | AGGTTCCCTG | |
| | GG | | | | |
| PDCD1_51 | AGGGTTTGGA | 50 | PDCD1_56 | GACGTTACCT | 80 |
| | ACTGGCCGGC | | | CGTGCGGCC | |
| PDCD1_52 | GGTGCTGCT | 51 | PDCD1_57 | CTCTCTTTGAT | 81 |
| | AGTCTGGGT | | | CTGCGCCT | |
| | CC | | | | |
| PDCD1_53 | GCTTGTCCG | 52 | PDCD1_58 | GTTGGGCAGT | 82 |
| | TCTGGTTGC | | | TGTGTGACA | |
| | TG | | | | |
| PDCD1_54 | AGCTTGTCC | 53 | PDCD1_59 | AGCTTGTCCG | 83 |
| | GTCTGGTTG | | | TCTGGTTGC | |
| | CT | | | | |
| PDCD1_55 | GACGTTACC | 54 | PDCDl_60 | CCTTCGGTCA | 84 |
| | TCGTGCGGC | | | CCACGAGCA | |
| | CC | | | | |

TABLE of
primers used for each gene:

| | | SEQ | | SEQ |
|---|---|---|---|---|
| | Primer- | ID | Primer- | ID |
| Gene | Forward | NO | Reverse | NO |
| CTLA4: | AAAGTCCTTGAT | 85 | AGGCATTC | 86 |
| 1, 2, | TCTGTGTGGGT | | TTCCCACA | |
| 3, 4, 5 | | | ATTTCCC | |
| CTLA4: | TAGAAGGCAGA | 87 | GGTTAGCACT | 88 |
| 6, 7, 8, 9, | AGGGCTTGC | | CCAGAGCGAG | |
| 10 | | | | |
| PTPN2: 20 | TGGCTGACCAT | 89 | ATATCCAAAGC | 90 |
| | AGATACCTCCA | | CACTGTCAAAG | |
| | | | C | |
| PTPN2: | GTCACAATGGC | 91 | AGAAGCATAAG | 92 |
| 11, 15, 19 | TAATGTGCTACA | | CAGCACTCTGT | |
| | A | | | |
| PTPN2: | GGTTCCTACCCA | 93 | TCTTGGAGATG | 94 |
| 14, 18 | AGTTTGTCTCT | | AAAGGTCTGCA | |
| | | | A | |
| PTPN2: | GGGATTGTCAG | 95 | AGCTACCAGGA | 96 |
| 16, 17 | AAAACAAATGG | | AGAAAAACACC | |
| | AAA | | T | |
| DGKB: | GGTTGACCACC | 97 | TGGAGAGCCTC | 98 |
| 21, 22 | AATTTTCCCTT | | TTGCTTTAGAT | |
| | AT | | | |
| DGKB: | CATGACGATGG | 99 | GCTGAAGACTT | 100 |
| 28, 29 | CTTGGGGTA | | GGAAAATGTCC | |
| | | | TT | |
| DGKB: 25, | CACCAAGCCATT | 101 | CACGTCTTC | 102 |
| 26, 27 | TGGCAGTC | | AGTGTGGGT | |
| | | | GA | |
| DGKB: | GTCACAGAAGC | 103 | GCATCTCCAGC | 104 |
| 23, 24 | TGCTAGATGGT | | AAAATTGCCC | |
| HAVCR2: | AGCGAATCATC | 105 | TGGGGCCTGTT | 106 |
| 41, 42, 44, | CTCCAAACAG | | AAACTTTAGGT | |
| 45, 47, | | | | |
| 48, 49, 50 | | | | |
| HAVCR2: | TTGTGTGGCTGT | 107 | CCAGTCCAGGG | 108 |
| 43, 46 | TAGTTCCGC | | TCAGTCAGAA | |
| DGKB: 30 | GAACCCCCTAA | 109 | TTTTAGCTGC | 110 |
| | CAGAGACCC | | CATAGGGTGG | |
| | | | TC | |
| PTPN2: | CAGCGCTCTCCC | 111 | GCCCCGAGCGA | 112 |
| 12, 13 | CGGATCG | | GAGGCTAGA | |
| LAG3: 32 | GCAGCCGCTTT | 113 | GCAAGCGAGGG | 114 |
| | GGGTGGCTC | | CAGGGAGACT | |
| LAG3: | ACACCCGTGCC | 115 | CGTGCTTCGGG | 116 |
| 31, 33, 34, | GGTCCTCTG | | GGCACCTTC | |
| 35, 36, 37 | | | | |
| LAG3: | CCAGTGGGCTG | 117 | CCCACAGCAAT | 118 |
| 38, 39, 40 | ATGAAGTCT | | GACGTAGGC | |
| PDCD1: | GGGTGAGCTGAG | 119 | GTGCGCCTG | 120 |
| 53, 54, 57, | CCGGTCC | | GCTCCTATT | |
| 58, 59, 60 | | | GTCCC | |
| PDCD1: 51, | CTCTGTATTCC | 121 | CAGTGAAAT | 122 |
| 52, 55, 56 | AGGGCCAGC | | GGCTTTGC | |
| | | | TCA | |

Therefore the method of the invention is capable of enabling efficient editing of patient derived T-cells while reducing mosaicism.

The inventors have demonstrated the ability to control mosaicism through rational design of guide RNA. Advantageously, this allows for direct creation of animals with homogenous edits throughout all tissues and with the ability to pass engineered edits to the next generation. This method can be used to rapidly create experiment-ready mouse models of disease in a fraction of the time and using minimal amounts of animals. The inventors have further demonstrated the ability to control mosaicism in human cells of therapeutic significance. Specifically the inventors have used the method herein to edit primary human T-cells in a controlled manner, thereby rapidly creating homogenous populations of cells which can be used directly for therapy.

In addition, the inventors have demonstrated the ability, not only to create homogenous edits, but also to use the method to create desired large deletions in mice by targeting regions of low microhomology. Thereby providing an efficient alternative approach to generate gene knockout models.

1. A method of selecting one or more guide RNA sequences for use in CRISPR-Cas editing of a nucleic acid sequence, the method comprising:
identifying a plurality of guide RNA sequences which target the nucleic acid sequence;
determining the frequency of editing outcomes for each of the plurality of guide RNA sequences; and
selecting one or more guide RNA sequences for which the frequency of the most abundant editing outcome is determined to be at least 2-fold greater than the frequency of the second most abundant editing outcome.

2. The method according to claim 1, wherein the frequency of editing outcomes for each of the plurality of guide RNA sequences are determined using a computer model.

3. The method according to claim 1 or claim 2, wherein the nucleic acid sequence is a gene sequence and the method further comprises, prior to identifying the plurality of guide RNA sequences, identifying the primary transcript(s) of the gene.

4. The method according to any preceding claim, further comprising selecting the guide RNA sequences which target a region located in the first approximately 50% of a gene.

5. The method according to any preceding claim, further comprising excluding any guide RNA sequences which target orphan exons that are not present in all major transcripts of a gene.

6. The method according to any preceding claim, wherein the method further comprises selecting the guide RNA sequences which are predicted to result in a frameshifting mutation.

7. The method according to any preceding claim, further comprising assigning each guide RNA sequence an off-target score and excluding any guide RNA sequences with a score below a predetermined threshold.

8. The method according to any preceding claim, further comprising assigning each guide RNA sequence an on-target activity score on-target activity score, and excluding any guide RNA sequences with a score below a predetermined threshold.

9. The method according to any preceding claim, further comprising generating a guide RNA molecule comprising a guide RNA sequence selected using the method of any one of claims 1 to 8.

10. The method according to claim 9, wherein the guide RNA molecule is a single guide RNA.

11. The method according to any preceding claim, further comprising using one or more guide RNA molecules, comprising one or more guide RNA sequences selected, to edit target sequences in a test population of cells, and determining the editing outcomes associated with each guide RNA sequence in the cells.

12. The method according to claim 11 further comprising selecting from the one or more guide RNA molecules those guide RNA molecules that most consistently cause the predicted most abundant outcome in cells of the test population.

13. A method of selecting a pair of guide RNA sequences for use in CRISPR-Cas editing of a nucleic acid sequence, the method comprising:
identifying a plurality of guide RNA sequences which target the 5′ and 3′ flanks surrounding the nucleic acid sequence;
determining the frequency of editing outcomes for each of the plurality of guide RNA sequences; and
selecting a pair of guide RNA sequences comprising a first guide RNA which targets the 5′ flank and a second guide RNA which targets the 3′ flank, wherein for each guide RNA the frequency of the most abundant editing outcome is determined to be less than 4 fold greater than the frequency of the second most abundant editing outcome.

14. A method according to claim 13, further comprising any of the features of claims 2-12.

15. A method for editing a nucleic acid sequence in an organism, a cell or a population of cells, or in a cell-free expression system, the method comprising exposing double-stranded (dsDNA) comprising the nucleic acid sequence to a Cas endonuclease and a guide RNA molecule which is capable of directing the Cas endonuclease to the target sequence within the nucleic acid sequence, wherein the guide RNA molecule comprises a guide RNA sequence which, when used in CRISPR-Cas editing, results in (or is predicted to result in, e.g. by a computer model), a major editing outcome having a frequency which is at least 2-fold greater than the second most abundant editing outcome.

16. The method according to claim 13, wherein the guide RNA molecule comprises a guide RNA sequence selected according to a method of any one of claims 1 to 12.

17. The method according to claim 13 or claim 14, further comprising introducing the guide RNA molecule and the DNA endonuclease into the cell or cells.

18. The method according to any one of claims 1 to 15, wherein the Cas endonuclease cleaves a target sequence within the nucleic acid sequence so as to produce a double strand break.

19. The method according to claim 16, wherein the Cas endonuclease is a Cas9 endonuclease.

20. The method according to any one of claims 13 to 17, wherein the organism, cell or population of cells is eukaryotic.

21. The method according to claim 18, wherein the organism, cell or population of cells is from an animal, fungus or plant, preferably the organism, cell or population of cells is mammalian.

22. The method according to claim 19, wherein the cell is a zygote or the population of cells form a zygote.

23. The method according to claim 20, wherein the method further comprises transferring the embryo into a recipient female animal for gestation, optionally wherein the embryo is cultured to a later stage of development prior to transfer.

24. The method according to any one of claims 13 to 21, wherein the method is for generating a non-mosaic transgenic animal.

25. The method according to claim 22, wherein the animal is a rodent, a rabbit, sheep, goat, horse, cow, pig, dog, cat, chicken, or a primate.

26. A method for editing a nucleic acid sequence in an organism, a cell or a population of cells, or in a cell-free expression system, the method comprising exposing double-stranded (dsDNA) comprising the nucleic acid sequence to a Cas endonuclease and a pair of guide RNA molecules which are capable of directing the Cas endonuclease to target the 5′ and 3′ flanks surrounding the nucleic acid sequence, wherein the pair of guide RNA molecules comprises a first guide RNA and a second guide RNA which, when used in CRISPR-Cas editing, result in (or are predicted to result in, e.g. by a computer model), a major editing outcome having a frequency which is less than 4 fold greater than the frequency of the second most abundant editing outcome.

27. A method according to claim 26 further comprising any of the features of claims 16-25.

28. Cells, cell populations and non-human organisms obtained by the methods of any one of claims 15-27.


SEQUENCE LISTING (122 sequences)

SEQ ID NO: 1 - DNA, 20 bp, Artificial Sequence
  Vsig4 guide
atgatcccctgagaggctac

SEQ ID NO: 2 - DNA, 20 bp, Artificial Sequence
  Vsig4 Forward Primer
cctaactctcacataatatt

SEQ ID NO: 3 - DNA, 20 bp, Artificial Sequence
  Vsig4 Reverse Primer
attacagagaacctatgtac

SEQ ID NO: 4 - DNA, 20 bp, Artificial Sequence
  Ccr1 sgRNA
ctctctgggttttattacct

SEQ ID NO: 5 - DNA, 20 bp, Artificial Sequence
  Prdm14 sgRNA
ggtcaatgccagcgaagtga

SEQ ID NO: 6 - DNA, 24 bp, Artificial Sequence
  Ccr1 Forward Primer
atggagatttcagatttcacagaa

SEQ ID NO: 7 - DNA, 21 bp, Artificial Sequence
  Ccr1 Reverse Primer
ccttccttctcactgggtctt

SEQ ID NO: 8 - DNA, 20 bp, Artificial Sequence
  Prdm14 Forward Primer
taaatcctctctagggactg

SEQ ID NO: 9 - DNA, 20 bp, Artificial Sequence
  Prdm14 Reverse Primer
tttcctgtagcatgctttta

SEQ ID NO: 10 - DNA, 20 bp, Artificial Sequence
  DDX3y_5′_HMH sgRNA
tccagtgtctatcactgtac

SEQ ID NO: 11 - DNA, 20 bp, Artificial Sequence
  DDX3y_3′_HMH sgRNA
tagtaaattcttaggtaagt

SEQ ID NO: 12 - DNA, 20 bp, Artificial Sequence
  DDX3y_5′_LMH sgRNA
cccagtacagtgatagacac

SEQ ID NO: 13 - DNA, 20 bp, Artificial Sequence
  DDX3y_3′_LMH sgRNA
aatcttaacttagcaaagtc

SEQ ID NO: 14 - DNA, 20 bp, Artificial Sequence
  Gata1_5′_HMH sgRNA
gccgcagtaacaggctgtct

SEQ ID NO: 15 - DNA, 20 bp, Artificial Sequence
  Gata1_3′_HMH sgRNA
acgccagctctggcctgctc

SEQ ID NO: 16 - DNA, 20 bp, Artificial Sequence
  Gata1_5′_LMH sgRNA
ctgtcttggggctggggggc

SEQ ID NO: 17 - DNA, 20 bp, Artificial Sequence
  Gata1_3′_LMH sgRNA
ccagagctggcgtaagcccc

SEQ ID NO: 18 - DNA, 20 bp, Artificial Sequence
  Gata1 Forward Primer
tgtccctgctgctttctgtc

SEQ ID NO: 19 - DNA, 20 bp, Artificial Sequence
  Gata 1 Reverse Primer
gttggacctgtatgcgcgtg

SEQ ID NO: 20 - DNA, 24 bp, Artificial Sequence
  DDX3y Forward Primer
taccaagccacatttgtagctccc

SEQ ID NO: 21 - DNA, 22 bp, Artificial Sequence
  DDX3y Reverse Primer
aatccgggccacagcttcttgt

SEQ ID NO: 22 - DNA, 20 bp, Artificial Sequence
  CTLA4_intron sgRNA
tgaggatctggataactaag

SEQ ID NO: 23 - DNA, 20 bp, Artificial Sequence
  CTLA4_intron Forward Primer
ctctgtattccagggccagc

SEQ ID NO: 24 - DNA, 20 bp, Artificial Sequence
  CTLA4_intron Reverse Primer
cagtgaaatggctttgctca

SEQ ID NO: 25 - DNA, 20 bp, Artificial Sequence
  CTLA4_1 sgRNA
cataaagccatggcttgcct

SEQ ID NO: 26 - DNA, 20 bp, Artificial Sequence
  CTLA4_2 sgRNA
tgaacctggctaccaggacc

SEQ ID NO: 27 - DNA, 20 bp, Artificial Sequence
  CTLA4_3 sgRNA
ctcagctgaacctggctacc

SEQ ID NO: 28 - DNA, 20 bp, Artificial Sequence
  CTLA4_4 sgRNA
agggccaggtcctggtagcc

SEQ ID NO: 29 - DNA, 20 bp, Artificial Sequence
  CTLA4_5 sgRNA
ccttggatttcagcggcaca

SEQ ID NO: 30 - DNA, 20 bp, Artificial Sequence
  PTPN2_11 sgRNA
ctcttctatgtcaactaaac

SEQ ID NO: 31 - DNA, 20 bp, Artificial Sequence
  PTPN2_12 sgRNA
catgcccaccaccatcgagc

SEQ ID NO: 32 - DNA, 20 bp, Artificial Sequence
  PTPN2_13 sgRNA
ctcttcgaactcccgctcga

SEQ ID NO: 33 - DNA, 20 bp, Artificial Sequence
  PTPN2_14 sgRNA
gttcagcatgacaactgctt

SEQ ID NO: 34 - DNA, 20 bp, Artificial Sequence
  PTPN2_15 sgRNA
ttgacatagaagaggcacaa

SEQ ID NO: 35 - DNA, 20 bp, Artificial Sequence
  DGKB_21 sgRNA
tctctggaggaatggattca

SEQ ID NO: 36 - DNA, 20 bp, Artificial Sequence
  DGKB_22 sgRNA
ctggaggaatggattcaagg

SEQ ID NO: 37 - DNA, 20 bp, Artificial Sequence
  DGKB_23 sgRNA
ggtaaaatatggtccttcaa

SEQ ID NO: 38 - DNA, 20 bp, Artificial Sequence
  DGKB_24 sgRNA
atgtgactgtggacctttga

SEQ ID NO: 39 - DNA, 20 bp, Artificial Sequence
  DGKB_25 sgRNA
ggcacttatcacacttggtt

SEQ ID NO: 40 - DNA, 20 bp, Artificial Sequence
  Lag3_31 sgRNA
cgccggcgagtaccgcgccg

SEQ ID NO: 41 - DNA, 20 bp, Artificial Sequence
  Lag3_32 sgRNA
ggctgaggtcccggtggtgt

SEQ ID NO: 42 - DNA, 20 bp, Artificial Sequence
  Lag3_33 sgRNA
aggagggcgccgccgggtga

SEQ ID NO: 43 - DNA, 20 bp, Artificial Sequence
  Lag3_34 sgRNA
cgctatggctgcgcccagcc

SEQ ID NO: 44 - DNA, 20 bp, Artificial Sequence
  Lag3_35 sgRNA
ccctgaggtgcaccgcggcg

SEQ ID NO: 45 - DNA, 20 bp, Artificial Sequence
  HAVCR2_41 sgRNA
aatgtgactctagcagacag

SEQ ID NO: 46 - DNA, 20 bp, Artificial Sequence
  HAVCR2_42 sgRNA
tgtgtttgaatgtggcaacg

SEQ ID NO: 47 - DNA, 20 bp, Artificial Sequence
  HAVCR2_43 sgRNA
tctctgccgagtcggtgcag

SEQ ID NO: 48 - DNA, 20 bp, Artificial Sequence
  HAVCR2_44 sgRNA
ggtgtagaagcagggcagat

SEQ ID NO: 49 - DNA, 20 bp, Artificial Sequence
  HAVCR2_45 sgRNA
agaagtggaatacagagcgg

SEQ ID NO: 50 - DNA, 20 bp, Artificial Sequence
  PDCD1_51 sgRNA
agggtttggaactggccggc

SEQ ID NO: 51 - DNA, 20 bp, Artificial Sequence
  PDCD1_52 sgRNA
ggtgctgctagtctgggtcc

SEQ ID NO: 52 - DNA, 20 bp, Artificial Sequence
  PDCD1_53 sgRNA
gcttgtccgtctggttgctg

SEQ ID NO: 53 - DNA, 20 bp, Artificial Sequence
  PDCD1_54 sgRNA
agcttgtccgtctggttgct

SEQ ID NO: 54 - DNA, 20 bp, Artificial Sequence
  PDCD1_55 sgRNA
gacgttacctcgtgcggccc

SEQ ID NO: 55 - DNA, 19 bp, Artificial Sequence
  CTLA4_6 sgRNA
tccatgctagcaatgcacg

SEQ ID NO: 56 - DNA, 19 bp, Artificial Sequence
  CTLA4_7 sgRNA
cacaaagctggcgatgcct

SEQ ID NO: 57 - DNA, 19 bp, Artificial Sequence
  CTLA4_8 sgRNA
ctgccgaagcactgtcacc

SEQ ID NO: 58 - DNA, 19 bp, Artificial Sequence
  CTLA4_9 sgRNA
tgtgcggcaacctacatga

SEQ ID NO: 59 - DNA, 19 bp, Artificial Sequence
  CTLA4_ 10 sgRNA
ttcacttgatttccactgg

SEQ ID NO: 60 - DNA, 19 bp, Artificial Sequence
  PTPN2_16 sgRNA
gtggatcaccgcaggccca

SEQ ID NO: 61 - DNA, 19 bp, Artificial Sequence
  PTPN2_17 sgRNA
gggactccaaaatctggcc

SEQ ID NO: 62 - DNA, 19 bp, Artificial Sequence
  PTPN2_18 sgRNA
cgcattgtggagaaagaat

SEQ ID NO: 63 - DNA, 19 bp, Artificial Sequence
  PTPN2_19 sgRNA
agtttagttgacatagaag

SEQ ID NO: 64 - DNA, 19 bp, Artificial Sequence
  PTPN2_20 sgRNA
catgactatcctcatagag

SEQ ID NO: 65 - DNA, 19 bp, Artificial Sequence
  DGKB_26 sgRNA
acataggtcttgatgcaag

SEQ ID NO: 66 - DNA, 19 bp, Artificial Sequence
  DGKB_27 sgRNA
tcgagccacacagcgctca

SEQ ID NO: 67 - DNA, 19 bp, Artificial Sequence
  DGKB_28 sgRNA
gaacatgctgattggcgtg

SEQ ID NO: 68 - DNA, 19 bp, Artificial Sequence
  DGKB_29 sgRNA
cgtcccatgcagaacgtga

SEQ ID NO: 69 - DNA, 19 bp, Artificial Sequence
  DGKB_30 sgRNA
tcgcctttatgacacggat

SEQ ID NO: 70 - DNA, 19 bp, Artificial Sequence
  Lag3_36 sgRNA
gctcatccagctggacgcg

SEQ ID NO: 71 - DNA, 19 bp, Artificial Sequence
  Lag3_37 sgRNA
gtcccgccccacatactcg

SEQ ID NO: 72 - DNA, 19 bp, Artificial Sequence
  Lag3_38 sgRNA
tgcattggttccggaaccg

SEQ ID NO: 73 - DNA, 19 bp, Artificial Sequence
  Lag3_39 sgRNA
atggggggactcccggaca

SEQ ID NO: 74 - DNA, 19 bp, Artificial Sequence
  Lag3_40 sgRNA
gaggaagctttccgctaag

SEQ ID NO: 75 - DNA, 19 bp, Artificial Sequence
  HAVCR2_46 sgRNA
ctctctgccgagtcggtgc

SEQ ID NO: 76 - DNA, 19 bp, Artificial Sequence
  HAVCR2_47 sgRNA
atgtgactctagcagacag

SEQ ID NO: 77 - DNA, 19 bp, Artificial Sequence
  HAVCR2_48 sgRNA
taaatggggatttccgcaa

SEQ ID NO: 78 - DNA, 19 bp, Artificial Sequence
  HAVCR2_49 sgRNA
gtgtttgaatgtggcaacg

SEQ ID NO: 79 - DNA, 19 bp, Artificial Sequence
  HAVCR2_50 sgRNA
acgggcacgaggttccctg

SEQ ID NO: 80 - DNA, 19 bp, Artificial Sequence
  PDCD1_56 sgRNA
gacgttacctcgtgcggcc

SEQ ID NO: 81 - DNA, 19 bp, Artificial Sequence
  PDCD1_57 sgRNA
ctctctttgatctgcgcct

SEQ ID NO: 82 - DNA, 19 bp, Artificial Sequence
  PDCD1_58 sgRNA
gttgggcagttgtgtgaca

SEQ ID NO: 83 - DNA, 19 bp, Artificial Sequence
  PDCD1_59 sgRNA
agcttgtccgtctggttgc

SEQ ID NO: 84 - DNA, 19 bp, Artificial Sequence
  PDCD1_60 sgRNA
ccttcggtcaccacgagca

SEQ ID NO: 85 - DNA, 23 bp, Artificial Sequence
  CTLA4 1,2,3,4,5 Forward Primer
aaagtccttgattctgtgtgggt

SEQ ID NO: 86 - DNA, 23 bp, Artificial Sequence
  CTLA4 1,2,3,4,5 Reverse Primer
aggcattcttcccacaatttccc

SEQ ID NO: 87 - DNA, 20 bp, Artificial Sequence
  CTLA4 6,7,8,9,10 Forward Primer
tagaaggcagaagggcttgc

SEQ ID NO: 88 - DNA, 20 bp, Artificial Sequence
  CTLA4 6,7,8,9,10 Reverse Primer
ggttagcactccagagcgag

SEQ ID NO: 89 - DNA, 22 bp, Artificial Sequence
  PTPN2 20 Forward Primer
tggctgaccatagatacctcca

SEQ ID NO: 90 - DNA, 23 bp, Artificial Sequence
  PTPN2 20 Reverse Primer
atatccaaagccactgtcaaagc

SEQ ID NO: 91 - DNA, 24 bp, Artificial Sequence
  PTPN2 11,15,19 Forward Primer
gtcacaatggctaatgtgctacaa

SEQ ID NO: 92 - DNA, 22 bp, Artificial Sequence
  PTPN2 11,15,19 Reverse Primer
agaagcataagcagcactctgt

SEQ ID NO: 93 - DNA, 23 bp, Artificial Sequence
  PTPN2 14,18 Forward Primer
ggttcctacccaagtttgtctct

SEQ ID NO: 94 - DNA, 23 bp, Artificial Sequence
  PTPN2 14,18 Reverse Primer
tcttggagatgaaaggtctgcaa

SEQ ID NO: 95 - DNA, 25 bp, Artificial Sequence
  PTPN2 16,17 Forward Primer
gggattgtcagaaaacaaatggaaa

SEQ ID NO: 96 - DNA, 23 bp, Artificial Sequence
  PTPN2 16,17 Reverse Primer
agctaccaggaagaaaaacacct

SEQ ID NO: 97 - DNA, 24 bp, Artificial Sequence
  DGKB 21,22 Forward Primer
ggttgaccaccaattttcccttat

SEQ ID NO: 98 - DNA, 22 bp, Artificial Sequence
  DGKB 21,22 Reverse Primer
tggagagcctcttgctttagat

SEQ ID NO: 99 - DNA, 20 bp, Artificial Sequence
  DGKB 28,29 Forward Primer
catgacgatggcttggggta

SEQ ID NO: 100 - DNA, 24 bp, Artificial Sequence
  DGKB 28,29 Reverse Primer
gctgaagacttggaaaatgtcctt

SEQ ID NO: 101 - DNA, 20 bp, Artificial Sequence
  DGKB 25,26,27 Forward Primer
caccaagccatttggcagtc

SEQ ID NO: 102 - DNA, 20 bp, Artificial Sequence
  DGKB 25,26,27 Reverse Primer
cacgtcttcagtgtgggtga

SEQ ID NO: 103 - DNA, 22 bp, Artificial Sequence
  DGKB 23,24 Forward Primer
gtcacagaagctgctagatggt

SEQ ID NO: 104 - DNA, 21 bp, Artificial Sequence
  DGKB 23,24 Reverse Primer
gcatctccagcaaaattgccc

SEQ ID NO: 105 - DNA, 21 bp, Artificial Sequence
  HAVCR2 41,42,44,45,47,48,49,50 Forward Primer
agcgaatcatcctccaaacag

SEQ ID NO: 106 - DNA, 22 bp, Artificial Sequence
  HAVCR2 41,42,44,45,47,48,49,50 Reverse Primer
tggggcctgttaaactttaggt

SEQ ID NO: 107 - DNA, 21 bp, Artificial Sequence
  HAVCR2 43,46 Forward Primer
ttgtgtggctgttagttccgc

SEQ ID NO: 108 - DNA, 21 bp, Artificial Sequence
  HAVCR2 43,46 Reverse Primer
ccagtccagggtcagtcagaa

SEQ ID NO: 109 - DNA, 20 bp, Artificial Sequence
  DGKB 30 Forward Primer
gaaccccctaacagagaccc

SEQ ID NO: 110 - DNA, 22 bp, Artificial Sequence
  DGKB 30 Reverse Primer
ttttagctgccatagggtggtc

SEQ ID NO: 111 - DNA, 19 bp, Artificial Sequence
  PTPN2 12,13 Forward Primer
cagcgctctccccggatcg

SEQ ID NO: 112 - DNA, 20 bp, Artificial Sequence
  PTPN2 12,13 Reverse Primer
gccccgagcgagaggctaga

SEQ ID NO: 113 - DNA, 20 bp, Artificial Sequence
  LAG3 32 Forward Primer
gcagccgctttgggtggctc

SEQ ID NO: 114 - DNA, 21 bp, Artificial Sequence
  LAG3 32 Reverse Primer
gcaagcgagggcagggagact

SEQ ID NO: 115 - DNA, 20 bp, Artificial Sequence
  LAG3 31,33,34,35,36,37 Forward Primer
acacccgtgccggtcctctg

SEQ ID NO: 116 - DNA, 20 bp, Artificial Sequence
  LAG3 31,33,34,35,36,37 Reverse Primer
cgtgcttcgggggcaccttc

SEQ ID NO: 117 - DNA, 20 bp, Artificial Sequence
  LAG3 38,39,40 Forward Primer
ccagtgggctgatgaagtct

SEQ ID NO: 118 - DNA, 20 bp, Artificial Sequence
  LAG3 38,39,40 Reverse Primer
cccacagcaatgacgtaggc

SEQ ID NO: 119 - DNA, 19 bp, Artificial Sequence
  PDCD1 53,54,57,58,59,60 Forward Primer
gggtgagctgagccggtcc

SEQ ID NO: 120 - DNA, 23 bp, Artificial Sequence
  PDCD1 53,54,57,58,59,60 Reverse Primer
gtgcgcctggctcctattgtccc

SEQ ID NO: 121 - DNA, 20 bp, Artificial Sequence
  PDCD1 51,52,55,56 Forward Primer
ctctgtattccagggccagc

SEQ ID NO: 122 - DNA, 20 bp, Artificial Sequence
  PDCD1 51,52,55,56 Reverse Primer
cagtgaaatggctttgctca